(19) United States
(12) Patent Application Publication
(10) Pub. No.: US 2024/0390284 A1
(43) Pub. Date: Nov. 28, 2024

(54) IONIZABLE LIPIDS

(71) Applicant: BRISTOL-MYERS SQUIBB COMPANY, Princeton, NJ (US)

(72) Inventors: Kendall Condon, Princeton, NJ (US); Donald Johnson, JR., Winchester, CA (US)

(73) Assignee: BRISTOL-MYERS SQUIBB COMPANY, Princeton, NJ (US)

(21) Appl. No.: 18/673,394

(22) Filed: May 24, 2024

Related U.S. Application Data
(60) Provisional application No. 63/504,257, filed on May 25, 2023.

(51) Int. Cl. A61K 9/127; A61K 31/7105; A61K 38/46; A61K 48/00; C07D 317/28; C07D 405/06

(52) U.S. Cl. CPC A61K 9/1272; A61K 31/7105; A61K 38/465; A61K 48/0033; C07D 317/28; C07D 405/06

(57) ABSTRACT

The application relates to ionizable lipids. The formulations of ionizable lipids can be used for the delivery of one or more nucleic acid therapeutic agents, for example mRNA, siRNA or miRNA. The compositions can include additional lipids, such as non-cationic lipids, PEG-modified lipids and optionally cholesterol.

### CROSS REFERENCE TO RELATED APPLICATIONS

This application claims the benefit of U.S. Provisional Application Ser. No. 63/504,257 filed May 25, 2023 which is incorporated herein in its entirety.

### FIELD

This invention relates to ionizable lipid compounds and their use in the formation of lipid nanoparticles.

### BACKGROUND OF THE INVENTION

Nucleic acids are useful for the treatment of various diseases and disorders. For example, RNA interference (RNAi) has been the subject of significant research and clinical development. Messenger RNA (mRNA) therapy is an important option for treatment of various diseases, in particular, for those associated with deficiency of one or more proteins. Small non-coding RNA, which regulates gene expression can be useful for the treatment of variety of diseases and disorders. Based on their biological roles and structures, small non-coding RNAs can be classified into three main categories: miRNAs, siRNAs, and piRNAs. In addition to therapeutic use, gene silencing by siRNA is an important tool to pinpoint the gene responsible for the specific pathological condition. RNA interference tools like siRNA can be used in studying the mammalian cellular signalling pathways. Sequence-specific binding of siRNA to the mRNA, and its site-specific cleavage results in the downregulation or inhibition of the genes responsible for cancer or other pathological conditions. However, there are several hurdles in the use of siRNA and mRNA in therapeutic setting which include degradation by the ribonucleases enzymes, stability of siRNA molecules in physiological conditions, inflammation reactions, site-specific and controlled release of siRNA, and efficient delivery vehicle. These barriers need to be overcome for the success of the use of mRNA and siRNA as therapeutics. Nitin Bharat Charbe, et. al., Small interfering RNA for cancer treatment: overcoming hurdles in delivery, Acta Pharmaceutica Sinica B, 10 (11) 2020, 2075-2109.

### SUMMARY

The present disclosure provides ionizable lipid compounds selected from Table 1:

TABLE 1

| Compound | |
|---|---|
| No. | Structure |
| 1. | 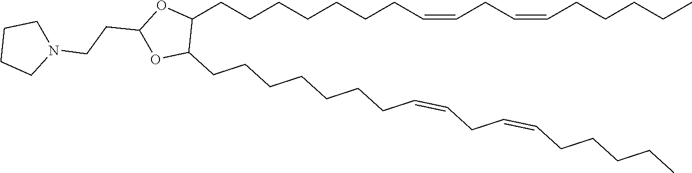 [SMILES: CCCCC/C=C\C/C=C\CCCCCCCC1OC(CCN2CCCC2)OC1CCCCCCC/C=C\C/C=C\CCCCC] |
| 2. | 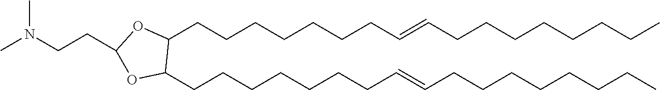 |
| 3. | 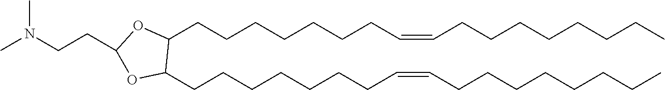 |
| 4. | 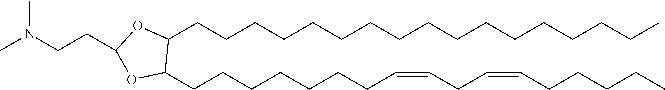 |
| 5. | 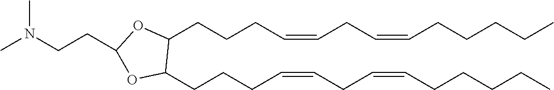 |
| 6. | 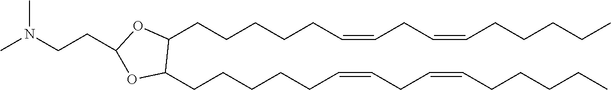 |
| 7. | 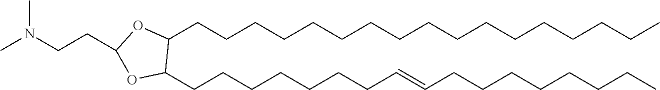 |
| 8. | 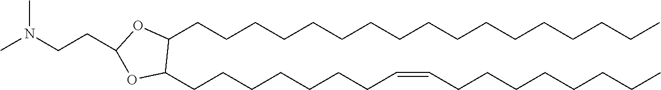 |
| 9. | 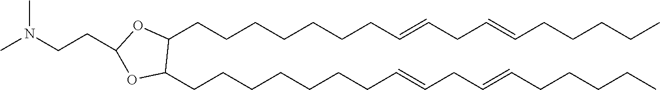 |
| 10. | 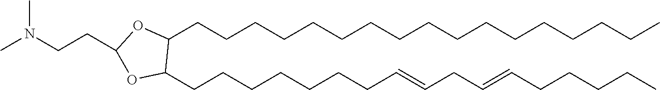 |

The application further provides pharmaceutical compositions comprising one or more ionizable lipids of Table 1. The formulations can be used for the delivery of one or more nucleic acid therapeutic agents, for example mRNA, siRNA or miRNA. The compositions can optionally include additional lipids, such as non-cationic lipids, PEG-modified lipids and optionally cholesterol.

### DETAILED DESCRIPTION

The present disclosure provides ionizable lipid compounds selected from Table 1:

TABLE 1

| Compound | |
|---|---|
| No. | Structure |
| 1. | 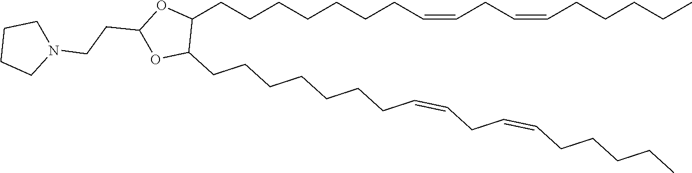 [SMILES: CCCCC/C=C\C/C=C\CCCCCCCC1OC(CCN2CCCC2)OC1CCCCCCC/C=C\C/C=C\CCCCC] |
| 2. | 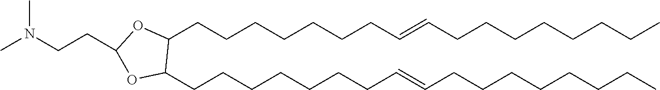 |
| 3. | 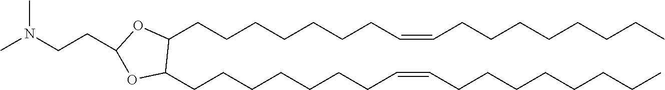 |
| 4. | 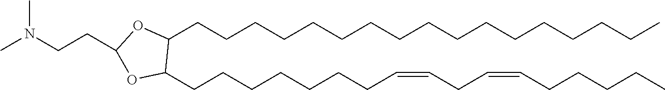 |
| 5. | 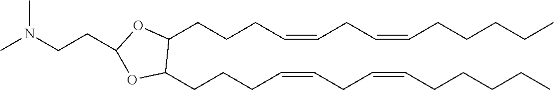 |
| 6. | 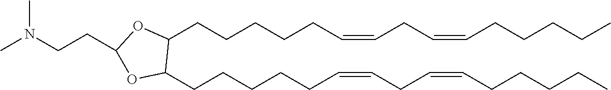 |
| 7. | 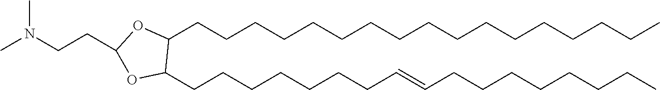 |
| 8. | 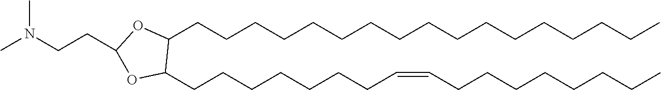 |
| 9. | 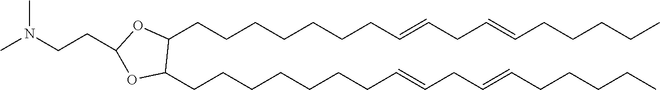 |
| 10. | 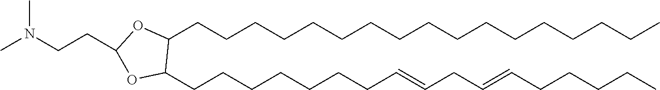 |

TABLE 2

| | |
|---|---|
| 11 | 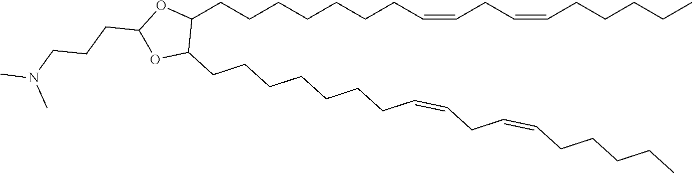 [SMILES: CCCCC/C=C\C/C=C\CCCCCCCC1OC(CCCN(C)C)OC1CCCCCCC/C=C\C/C=C\CCCCC] |
| 12 | 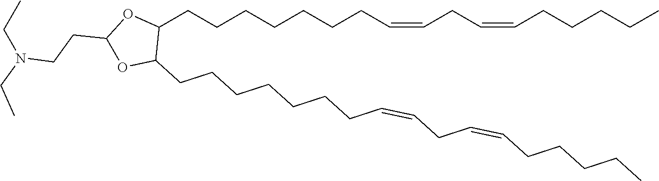 [SMILES: CCCCC/C=C\C/C=C\CCCCCCCC1OC(CCN(CC)CC)OC1CCCCCCC/C=C\C/C=C\CCCCC] |
| 13. | 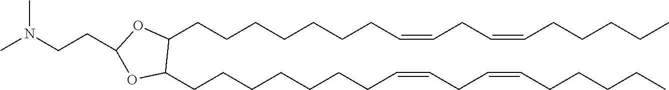 |

The application further provides pharmaceutical compositions comprising one or more ionizable lipids of Table 1 or 2. The formulations can be used for the delivery of one or more nucleic acid therapeutic agents, for example mRNA, siRNA or miRNA. The compositions can optionally include additional lipids, such as non-cationic lipids, PEG-modified lipids and optionally cholesterol.

In one aspect, provided is a method for encapsulating mRNA in lipid nanoparticles comprising the steps of mixing one or more lipids of Table 1 or 2, and optionally one or more additional lipids in a lipid solution with one or more mRNAs in an mRNA solution to form mRNA encapsulated within the LNP.

The following are definitions of terms used in this specification and appended claims. The initial definition provided for a group or term herein applies to that group or term throughout the specification and claims, individually or as part of another group, unless otherwise indicated.

In some embodiments, provided are lipid nanoparticle formulations comprising ionizable lipids of Table 1 or 2 and one or more mRNA moieties. In some embodiments, the mRNA is a modified mRNA. Modifications, for example, can improve resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozac consensus sequence). (Kozak, M., Nucleic Acids Res 15 (20): 8125-48 (1987)).

In one embodiment, provided are lipid nanoparticle compositions prepared to optimize delivery of the mRNA to a target cell. For example, if the target cell is a hepatocyte the properties of the lipid nanoparticles may be optimized to effectively deliver such transfer vehicle to the target cell, reduce immune clearance and/or promote retention in that target cell. In one embodiment, the compositions of the present invention may be combined with agents that facilitate the transfer of exogenous mRNA (e.g., agents which disrupt or improve the permeability of the blood brain barrier and thereby enhance the transfer of exogenous mRNA to the target cells).

The process of incorporation of a desired entity (e.g., a nucleic acid) into a lipid nanoparticle is often referred to as “loading” (Lasic, et al., FEBS Lett., 312:255-258, 1992). The LNP-incorporated nucleic acids may be completely or partially located in the interior space of the LNP, within the bilayer membrane of the LNP, or associated with the exterior surface of the LNP membrane. The purpose of incorporating a mRNA into a lipid nanoparticle is often to protect the nucleic acid from an environment which may contain enzymes or chemicals that degrade nucleic acids and/or systems or receptors that cause the rapid excretion of the nucleic acids. Accordingly, in a preferred embodiment of the present invention, the selected transfer vehicle is capable of enhancing the stability of the mRNA contained therein. The liposome can allow the encapsulated mRNA to reach the target cell and/or may preferentially allow the encapsulated mRNA to reach the target cell, or alternatively limit the delivery of such mRNA to other sites or cells where the presence of the administered mRNA may be useless or undesirable. Furthermore, incorporating the mRNA into a transfer vehicle, such as for example, a cationic liposome, also facilitates the delivery of such mRNA into a target cell.

Ideally, liposomal transfer vehicles are prepared to encapsulate one or more desired mRNA such that the compositions demonstrate a high transfection efficiency and enhanced stability. While liposomes can facilitate introduction of nucleic acids into target cells, the addition of polycations (e.g., poly L-lysine and protamine), as a copolymer can facilitate, and in some instances markedly enhance the transfection efficiency of several types of cationic liposomes by 2-28 fold in a number of cell lines both in vitro and in vivo. (See N.J. Caplen, ct al., Gene Ther. 1995; 2:603; S. Li, et al., Gene Ther. 1997; 4, 891.)

### Lipid Nanoparticles

In a preferred embodiment of the present invention, the transfer vehicle is formulated as a lipid nanoparticle. As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more mRNA to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a mRNA to a target cell.

As used in the present specification, the following words, phrases and symbols are generally intended to have the meanings as set forth below, except to the extent that the context in which they are used indicates otherwise.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis- and trans-geometric isomers of the compounds of the present invention are described and may be isolated as a mixture of isomers or as separated isomeric forms. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated.

The compounds of Table 1 or 2 may exist in a free form (with no ionization) or can form salts which are also within the scope of this invention. Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s)” may include zwitterions (inner salts), e.g., when a compound of Table 1, contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of herein may be formed, for example, by reacting a compound with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically-acceptable salts include, but are not limited to, mineral or organic acid salts of basic groups such as amines; and alkali or organic salts of acidic groups such as carboxylic acids. The pharmaceutically-acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, malcic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA (1990), the disclosure of which is hereby incorporated by reference.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

All stereoisomers of the compounds of the instant invention are contemplated, either in admixture or in pure or substantially pure form. Stereoisomers may include compounds which are optical isomers through possession of one or more chiral atoms, as well as compounds which are optical isomers by virtue of limited rotation about one or more bonds (atropisomers). The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The present invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

### Methods of Preparation

### EXAMPLES

The methods and conditions used in these examples, and the actual compounds prepared in these Examples, are not meant to be limiting, but are meant to demonstrate how the compounds can be prepared. Starting materials and reagents used in these examples, when not prepared by a procedure described herein, are generally either commercially available, or are reported in the chemical literature, or may be prepared by using procedures described in the chemical literature.

### Example 1: Synthesis of Lipid-1

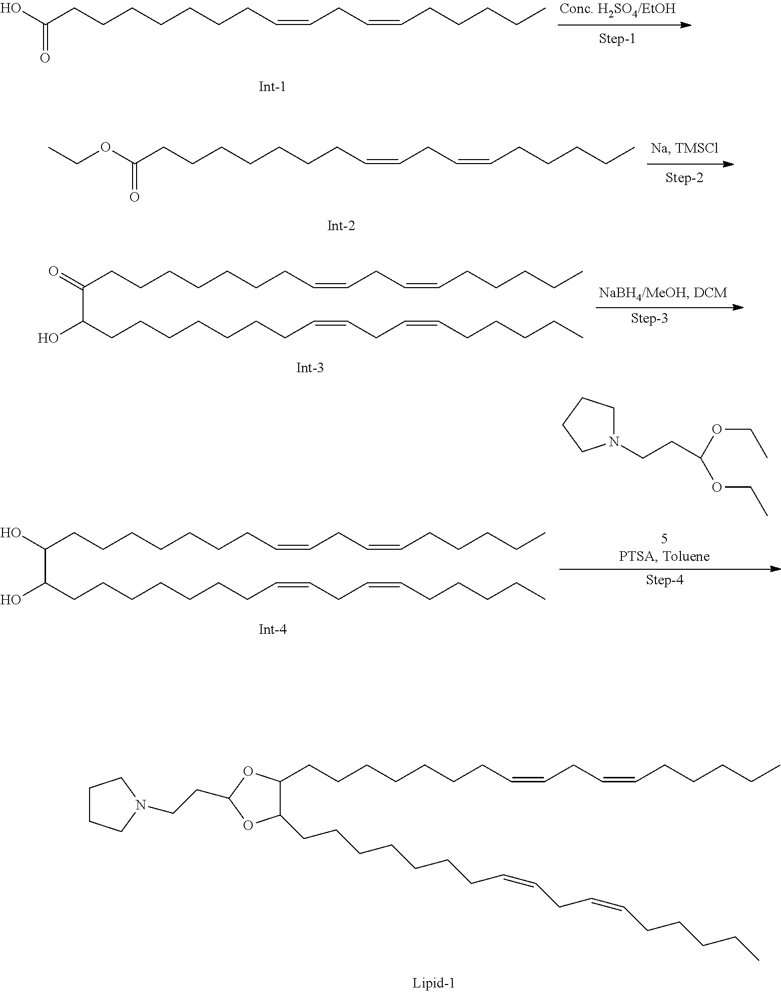

### Step-1: Synthesis of Ethyl (9Z,12Z)-Octadeca-9,12-Dienoate (Int-2)

Concentrated H₂SO₄ (0.1 V) was added to ethanol (250 mL, 5 V) at 0-5° C. A predissolved solution of Int-1 (50 g, 0.178 mol) in ethanol (250 mL, 5 V) was added drop wise to above mixture at 0-5° C. Resulting reaction mixture was refluxed at 80° C. for 18 h. Progress of the reaction was monitored by TLC. After completion of reaction, reaction mixture was cooled to 0° C. and pH was adjusted to ˜8 using 10% NaHCO₃ solution. Reaction mixture was evaporated to remove excess of ethanol and product was extracted with DCM (2×500 mL), organic layer was washed water, dried over Na₂SO₄ and concentrated under reduced pressure at 40° C. to afford crude Ethyl (9Z,12Z)-Octadeca-9,12-Dienoate (Int-2) as pale brown liquid. Yield: 53 g (crude); Characterized by 1H-NMR

### Step-2: Synthesis of (6Z,9Z,27Z,30Z)-19-Hydroxyhexatriaconta-6,9,27,30-Tetraen-18-One (Int-3)

To a round bottom flask (RB) containing sodium metal (14.92 g, 0.649 mol) and toluene (2.5 V), TMSCl (69.2 mL, 0.545 mol) was added drop wise at 25-30° C. Resulting reaction mixture was heated to 40° C. A predissolved solution of Int-2 (40 g, 0.129) in Toluene (6 V) was added dropwise at 40° C. After the complete addition, reaction mixture was refluxed at 115° C. for 5 h. Progress of the reaction was monitored by TLC, TLC shows complete consumption of Int-2. Reaction mixture was cooled to 0-5° C. and quenched by slowly adding methanol at 0-5° C. After quenching, reaction mixture was allowed to stir at 25-30° C. for 30 min. Reaction mixture was filtrate through celite bed, washed with MTBE (1000 mL). Combined filtrate was collected and stirred with sat. Ammonium chloride (500 mL) solution for 18 h. Organic layer was separated, dried over NaSO₄ and concentrated under reduced pressure at 40° C. to afford crude. Crude was purified through combi flash, compound was eluted at 3-4% ethyl acetate:hexane. Pure fractions were evaporated under reduced pressure at 40° C. to afford (6Z,9Z,27Z,30Z)-19-Hydroxyhexatriaconta-6,9,27,30-Tetraen-18-One (Int-3) as pale yellow liquid.

Yield: 10.8 g (15.7%); Characterized by ¹H-NMR

### Step-3: Synthesis of (6Z,9Z,27Z,30Z)-Hexatriaconta-6,9,27,30-Tetraene-18,19-Diol (Int-4)

To a cooled solution of the Int-3 (3.9 g, 0.0073 mol) in DCM (29.6 mL, 7.6 V) and methanol (29.6 mL, 7.6 V) was added NaHB₄ (0.418 g, 0.011 mol) at 0-5° C. Resulting reaction mixture was stirred at 0-5° C. for 30 min and then stirred at ambient temperature for 16 h. Progress of the reaction was monitored by TLC, TLC shows formation of new polar spot along with Int-3. Reaction mixture was quenched by adding purified water (30 mL) at 0-5° C. and product was extracted with DCM (3×50 mL), organic layer was dried over Na₂SO₄ and concentrated under reduced pressure at 40° C. to afford crude. Crude was purified through Combi flash, compound was eluted at 6% ethyl acetate:hexane. Pure fractions were evaporated under reduced pressure at 40° C. to afford (6Z,9Z, 27Z, 30Z)-Hexatriaconta-6,9,27,30-Tetraene-18,19-Diol (Int-4) as white semi solid.

Yield: 1.79 g (45.7%); Characterized by 1H-NMR

### Step-4: Synthesis of (3-{4,5-Bis [(8Z,11Z)-heptadeca-8,11-dien-1-yl]-1,3-Dioxolan-2-Yl}propyl)dimethylamine (Lipid-1)

To a stirred solution of Int-4 (750 mg, 0.0014 mol) and Int-6 (357 mg, 0.0017 mol) in toluene (50 mL), was added PTSA monohydride (338 mg, 0.0017 mol) at 25-30° C. Resulting reaction mixture was refluxed at 130° C. for 18 h in a dean-stark apparatus. Progress of the reaction was monitored by TLC. Reaction mixture was evaporated to remove toluene, residue obtained was diluted with ethyl acetate (60 mL), washed with sodium bicarbonate solution (20 mL). Organic layer was dried over Na₂SO₄ and concentrated under reduced pressure to afford crude. Crude was purified through Combi flash, compound was eluted at 3.5-4.5% methanol:DCM. Pure fractions were evaporated at 40° C. to get (3-{4,5-Bis [(8Z,11Z)-heptadeca-8,11-dien-1-yl]-1,3-Dioxolan-2-Y1}propyl)dimethylamine (Lipid-1) as pale yellow liquid.

Yield: 408 mg (45.0%); Characterized by 1H-NMR and LCMS

LCMS: (EI, m/z) calcd for C₄₃H₇₇NO₂ [M+H]: 640.9; HPLC Purity: 91.03%

1H NMR (400 MHZ, CDCl₃): 5.40-5.30 (8H, m), 5.20-4.95 (1H, m), 4.09-3.98 (1H, m), 3.52-3.62 (1H, m), 2.79-2.76 (6H, t, J=6.4 Hz), 2.08-2.03 (10H, m), 1.91 (4H, m), 1.51-1.34 (4H, m), 1.30-1.26 (36H, m), 0.89-0.88 (6H, t, J=3.6 Hz).

### Example 2: Synthesis of Lipid 2

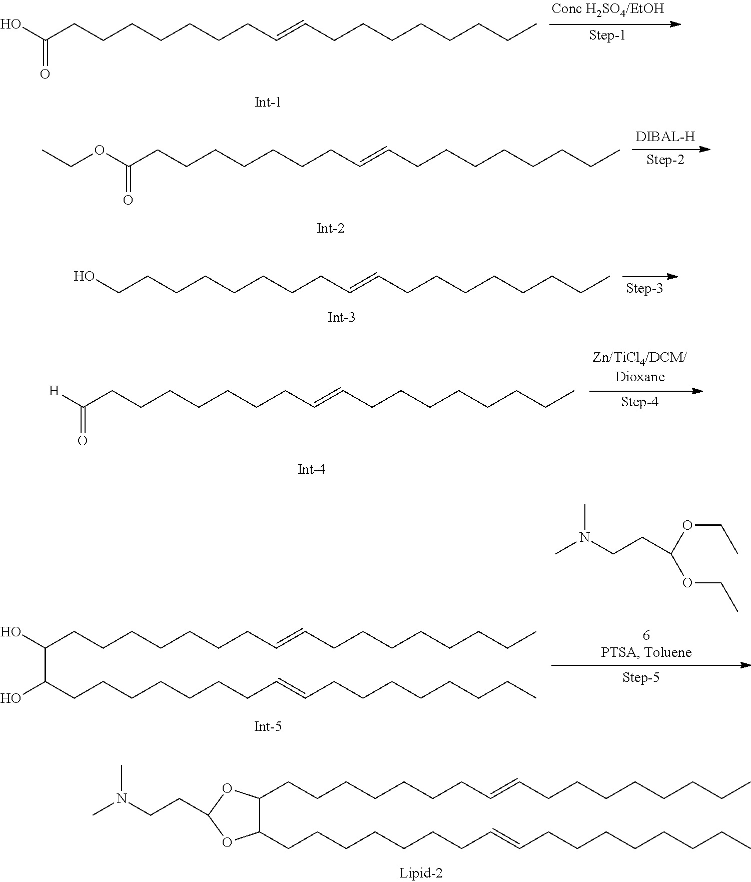

### Step-1: Synthesis of Ethyl (9E)-Octadec-9-enoate (Int-2)

Conc H₂SO₄ (0.5 mL, 0.1 V) was added dropwise to cold ethanol (50 mL, 10 V) at 0-5° C. A premixed solution of Int-1 (5.0 g, 0.017 mol) in ethanol (25 mL, 5 V) was added drop wise at 0-5° C. Resulting reaction mixture was refluxed at 80° C. for 18 h. Progress of the reaction was monitored by TLC, TLC of the reaction shows completion of the reaction. Reaction mixture was cooled to 0-5° C. and pH was adjusted to 8 using 10% NaHCO₃ solution. Reaction mixture was evaporated to remove excess of ethanol and extracted with dichloromethane (2×50 mL), organic layer was washed with water and dried over anhydrous sodium sulphate and evaporated at 40° C. to afford crude Ethyl (9E)-Octadec-9-enoate (Int-2) as pale brown liquid. Crude was used as such for the next reaction.

Yield: 5.0 g (crude); Characterized by 1H-NMR

### Step-2: Synthesis of (9E,12E)-octadeca-9,12-dien-1-ol (Int-3)

DiBAL-H (12.28 mL, 0.025, 2M in THF) was added slowly to a solution of Int-2 (4 g, 0.012 mol) in THF (40 mL, 10 V) at −72 to −75° C. over a period of 15 min. Resulting reaction mixture was allowed to stir at −72 to −75° C. for 2 h. Progress of the reaction was monitored by TLC. Reaction mixture was quenched by adding saturated ammonium chloride solution (30 mL) at 0-5° C., diluted with ethyl acetate (100 mL) and stirred at 25-30° C. for 30 min. Reaction mixture was filtered through celite bed. Filtrate was washed with water and extracted with ethyl acetate (3×50 mL). Combined organic layer was dried over anhydrous sodium sulphate and evaporated at 40° C. to afford crude (9E,12E)-octadeca-9,12-dien-1-ol (Int-3) as pale brown liquid. Crude was used as such for next reaction.

Yield: 3.2 g (crude); Characterized by ¹H-NMR

### Step-3: Synthesis of (9E)-octadec-9-enal (Int-4)

To an ice cold solution of Int-3 (3.2 g, 0.011 mol) in DCM (64 mL, 20 V) was added Dess-Martin periodinanc (5.56 g, 0.0114 mol) at 0-5° C. Reaction was allowed to stir at 25-30° C. for 1.5 h. Progress of the reaction was monitored by TLC. After 1.5 h TLC shows complete consumption of Int-3. Reaction mixture was cooled to 0-5° C. and quenched by adding saturated sodium bicarbonate solution (30 mL, 10 V) at 0-5° C. and product was extracted with DCM (3×50 mL). Combined organic layer was dried over anhydrous sodium sulphate and evaporated at 40° C. to get crude. Crude was purified by combi Flash® using 3 to 4% EtOAc in hexanes to afford (9E)-octadec-9-enal (Int-4) as colourless liquid.

Yield: 2.05 g (70.6%); Characterized by 1H-NMR

### Step-4: Synthesis of (9E,27E)-hexatriaconta-9,27-diene-18,19-diol (Int-5)

To a stirred solution of Zinc dust (1.33 g, 0.02 mol) in 1,4-Dioxane (15 mL, 7.5 V) and DCM (15 mL, 7.5 V) at 5-10° C., Titanium chloride (1.66 ml, 0.014 mol) was added over a period of 10 min. Reaction mixture was allowed to stir at 5-10° C. for 30 min. A predissolved solution of Int-4 (2.0 g, 0.07 mol) in 1,4 dioxane (20 mL, 10 V) was added to reaction mixture at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate was extracted with ethyl acetate (3×20 mL). Combined organic layer was dried over anhydrous sodium sulphate and evaporated at 40° C. to get crude. Crude was purified by combi Flash® using 6-8% ethyl acetate in hexanes to afford (9E,27E)-hexatriaconta-9,27-diene-18,19-diol (Int-5) as white semi solid.

Yield: 1.16 g (37%); Characterized by 1H-NMR

### Step-5: Synthesis of (2-{4,5-Bis [(8E)-heptadec-8-en-1-yl]-1,3-dioxolan-2-yl}ethyl)dimethylamine (Lipid-2)

To a solution of Int-5 (1.15 g, 0.0021 mol and Int-7 (470 mg, 0.0026 mol) in toluene (25 mL, 35 V), was added PTSA monohydride (514 mg, 0.0026 mol) at 25-30° C. Resulting reaction mixture was refluxed at 130° C. for 18 h in a dean-stark apparatus. Progress of the reaction was monitored by TLC, TLC shows completion of the reaction. The reaction mixture was evaporated to remove toluene, obtained residue was treated with sodium bicarbonate solution (20 mL) and product was extracted with Ethyl acetate (3×30 mL). Organic layer was dried over anhydrous sodium sulphate and evaporated at 40° C. to get crude. Crude was purified through combi flash, product was eluted at 25-45% Ethyl acetate: Hexane. Pure fractions were evaporated to afford (2-{4,5-Bis [(8E)-heptadec-8-en-1-yl]-1,3-dioxolan-2-yl}ethyl)dimethylamine (Lipid-2) as pale brown liquid.

Yield: 660 mg (49.6%); Characterized by 1H-NMR and LCMS

LCMS: (EI, m/z) calcd for C₄₁H₇₉NO₂ [M+H]: 618.8; HPLC Purity: 99.21%

¹H NMR (400 MHZ, CDCl₃): 5.43-5.34 (4H, m), 5.03-5.00 (1H, t, J=4.8 Hz), 4.02-3.50 (2H, m), 2.43-2.40 (2H, q, J₁=8.8 Hz, J₂=8.8 Hz), 2.24 (6H, s), 1.97-1.94 (2H, t, J=4.8 Hz), 1.85-1.82 (2H, q, J₁=8.8 Hz, J₂=8.8 Hz), 1.54-1.53 (6H, m), 1.34-1.18 (42H, m), 0.90-0.88 (6H, m);

### Example-3 Synthesis of Lipid-3

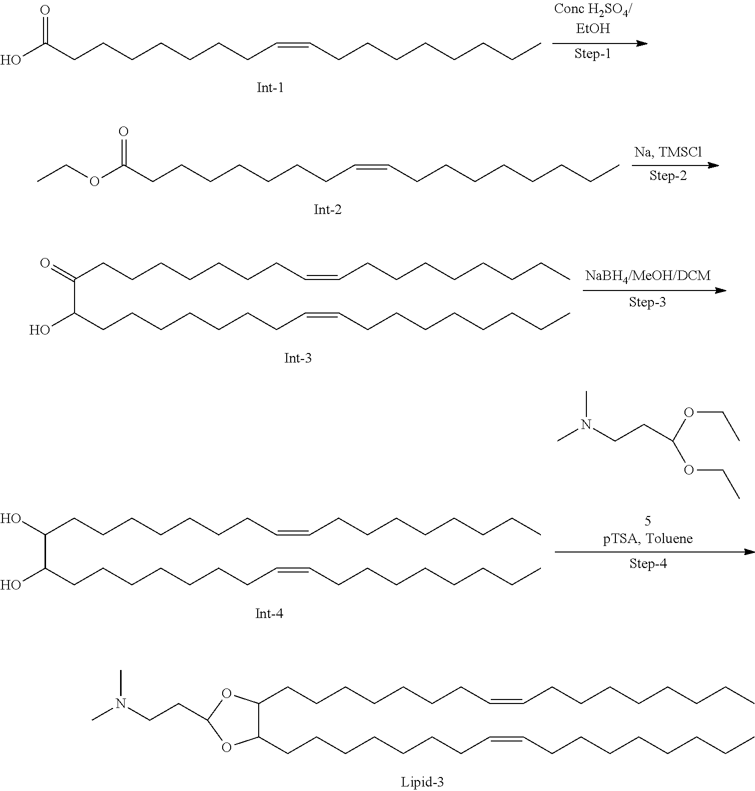

### Step-1: Synthesis of Ethyl (9Z)-Octadec-9-Enoate (Int-2)

Conc H₂SO₄ (0.5 mL, 0.1 V) was added dropwise to cold ethanol (15 mL, 5 V) at 0-5° C. A premixed solution of Int-1 (20 g, 0.0708 mol) in ethanol (100 mL, 5 V) was added drop wise at 0-5° C. Resulting reaction mixture was refluxed at 80° C. for 18 h. Progress of the reaction was monitored by TLC, TLC of the reaction shows completion of the reaction. Reaction mixture was cooled to 0-5° C. and pH was adjusted to 8 using 10% NaHCO₃ solution. Reaction mixture was evaporated to remove excess of ethanol and extracted with DCM (2×150 mL), organic layer was washed with water and dried over anhydrous sodium sulphate and evaporated at 40° C. to afford crude Ethyl (9Z)-Octadec-9-Enoate (Int-2) as a pale brown liquid. Crude was used as such for the next reaction.

Yield: 21 g (crude); Characterized by 1H-NMR

### Step-2: Synthesis of (9Z,27Z)-19-hydroxyhexatriaconta-9,27-dien-18-one (Int-3)

TMSCl (30.9 g, 0.143 mol) was added drop wise to a mixture of sodium metal (7.75 g, 0.17 mol) and toluene (2.5 V at 25-30° C. Reaction mixture was heated to 40° C. A premixed solution of Int-2 (21 g, 0.067) in Toluene (6 V) was added drop wise at 40° C., after the complete addition reaction mixture was refluxed at 115° C. for 18 h. Progress of the reaction was monitored by TLC, TLC shows complete consumption of Int-2. Reaction mixture was cooled to 0-5° C. and quenched by slowly adding methanol at 0-5° C. After quenching reaction mixture was allowed to stir at 25-30° C. for 30 min. Reaction mixture was filtrate through celite bed, and washed with MTBE (500 mL). Filtrate was collected and stirred with sat. Ammonium chloride (280 mL) solution for 18 h. Organic layer was separated, dried over anhydrous sodium sulphate and evaporated at 40° C. to get crude. Crude was purified through combi flash, compound was eluted at 1-1.5% ethyl acetate:hexane. Pure fractions were evaporated at 45° C. to afford (9Z,27Z)-19-hydroxyhexatriaconta-9,27-dien-18-one (Int-3) as pale yellow liquid.

Yield: 3.8 g (10.55%); Characterized by 1H-NMR

### Step-3: Synthesis of (9Z,27Z)-hexatriaconta-9,27-diene-18,19-diol (Int-4)

To a cooled solution of the Int-3 (3.8 g, 0.0071 mol) in dichloromethane (22.8 mL, 6 V) and methanol (22.8 mL, 6 V) was added NaBH₄ (0.404 g, 0.0106 mol) at 0-5° C. Reaction mixture was stirred at 0-5° C. for 30 min and then stirred at 25-30° C. for 18 h. Progress of the reaction was monitored by TLC, TLC shows formation of new polar spot along with unreacted Int-3. Reaction mixture was quenched by adding purified water at 0-5° C. and product was extracted with DCM (3×50 mL), organic layer was dried over anhydrous sodium sulphate and evaporated at 40° C. to afford crude. Crude was purified through combi flash, compound was eluted at 6-8% ethyl acetate:hexane. Pure fractions were evaporated to afford (9Z,27Z)-hexatriaconta-9,27-diene-18,19-diol (Int-4) as white semi solid.

Yield: 1.52 g (39.8%); Characterized by ¹H-NMR

### Step-4: Synthesis of (2-{4,5-Bis [(8Z)-heptadec-8-en-1-yl]-1,3-dioxolan-2-yl}ethyl)dimethylamine (Lipid-3)

To a solution of Int-4 (1.2 g, 0.0024 mol) and Int-5 (488 mg, 0.0028 mol) in toluene (50 mL, 50 V), was added PTSA monohydride (534 mg, 0.0028 mol) at 25-30° C. Resulting reaction mixture was refluxed at 130° C. for 18 h in a dean-stark apparatus. Progress of the reaction was monitored by TLC, TLC shows completion of the reaction. Reaction mixture was evaporated to remove toluene, obtained residue was treated with sodium bicarbonate solution and product was extracted with Ethyl acetate (3×50 mL). Organic layer was dried over anhydrous sodium sulphate and evaporated at 40° C. to get crude. Crude was purified through combi flash, product was eluted at 30-40% Ethyl acetate:Hexane. Pure fractions were evaporated to afford (2-{4,5-Bis [(8Z)-heptadec-8-en-1-yl]-1,3-dioxolan-2-yl}ethyl)dimethylamine (Lipid-3) as colourless liquid.

Yield: 650 mg (47.1%); Characterized by ¹H-NMR and LCMS

LCMS: (EI, m/z) calcd for C₄₁H₇₉NO₂ [M+H]: 620.8; HPLC Purity: 99.8%

¹H NMR (400 MHZ, CDCl₃): 5.40-5.30 (4H, m), 5.18-4.93 (1H, m), 4.02-3.55 (2H, m), 2.53 (2H, m), 2.32 (6H, s), 2.02-2.00 (12H, m), 1.30-1.27 (46H, m), 0.90-0.86 (6H, t, J=8.8 Hz).

### Example 4: Synthesis of Lipid-4

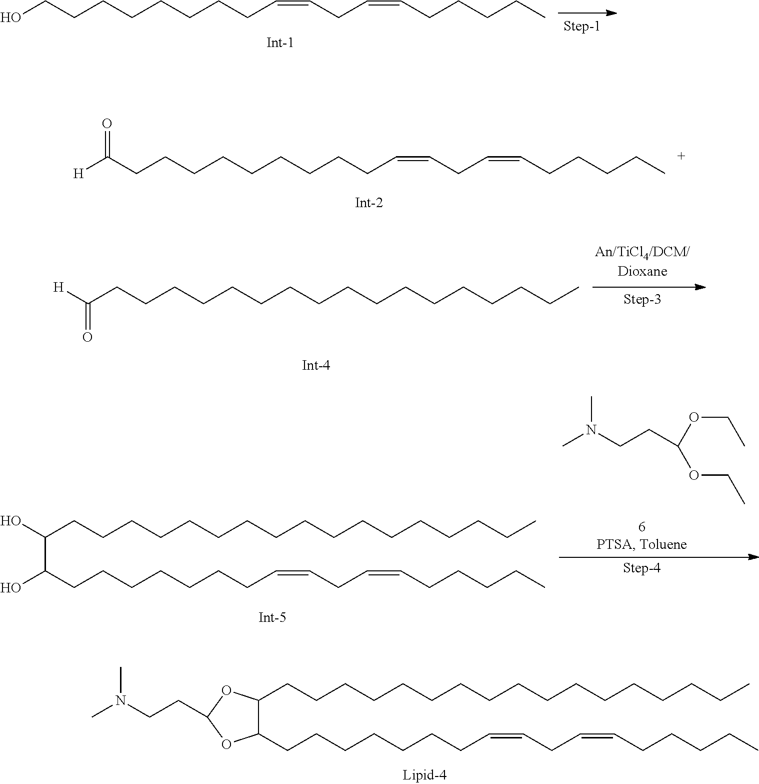

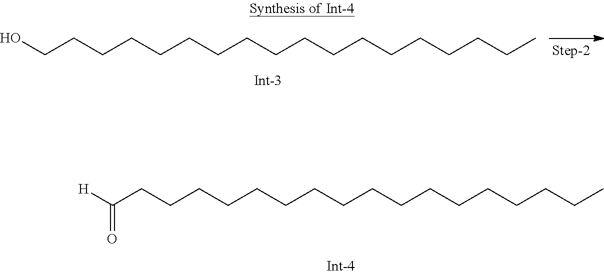 [SMILES: CCCCCCCCCCCCCCCCCCO ; CCCCCCCCCCCCCCCCCC=O]

### Step-1: Synthesis of (9Z,12Z)-octadeca-9,12-dienal (Int-2)

To an ice cold solution of Int-1 (10 g, 0.037 mol) in DCM (200 mL, 20 V) was added dessmortin periodinate (17.50 g, 0.041 mol) at 0-5° C. Resulting reaction mixture was allowed to attain 25-30° C. and stirred at same temperature for 1.5 h. Progress of the reaction was monitored by TLC, after 1.5 h TLC shows completion of reaction. Reaction mixture was quenched by adding 10% NaHCO₃ solution (100 mL, 10 V) at 0-5° C. and product was extracted with DCM (3×100 mL). Organic layer was dried over Na₂SO₄ and concentrated under reduced pressure to afford crude. Crude was purified through combi flash, compound was eluted at 3-4% ethyl acetate:hexane. Pure fractions were evaporated to get Ethyl (9Z,12Z)-octadeca-9,12-dienal (Int-2) as colourless liquid.

Yield: 6.8 g (68.8%); Characterized by 1H-NMR

### Step-2: Synthesis of Octadecanal (Int-4)

To an ice cold solution of Int-3 (15 g, 0.0554 mol) in DCM (300 mL, 20 V) was added dessmortin per iodinate (28.3 g, 0.066 mol) at 0-5° C. Reaction mixture was allowed to attain 25-30° C. and stirred at the same temperature for 1.5 h. Progress of the reaction was monitored by TLC, after 1.5 h TLC shows completion of the reaction. Reaction mixture was quenched by adding 10% NaHCO₃ solution (150 ml, 10 V) at 0-5° C. and product was extracted with DCM (3×150 mL). Organic layer was dried over Na₂SO₄ and concentrated under reduced pressure at 45° C. to get crude. Crude was purified through combi flash, compound was eluted at 3-4% ethyl acetate:hexane. Pure fractions were evaporated to afford Octadecanal (Int-4) as white solid.

Yield: 11.6 g (77.9%); Characterized by ¹H-NMR

### Step-3: Synthesis of (6Z,9Z)-hexatriaconta-6,9-diene-18,19-diol (Int-5)

To a stirred solution of Zinc dust (10.8 g, 0.166 mol) in 1,4-Dioxane (120 mL, 15 V) and DCM (120 mL, 15 V) at 5-10° C., Titanium chloride (132.5 mL, 0.121 mol) was added over a period of 10 min. Reaction mixture was allowed to stir at 5-10° C. for 30 min. A predissolved solution of Int-2 (8 g, 0.030 mol) and Int-4 (8.1 g, 0.030 mol) in 1,4 dioxane (120 mL, 15 V) was added to reaction mixture at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate was extracted with ethyl acetate (3×20 mL). Combined organic layer was dried over anhydrous sodium sulphate and evaporated at 40° C. to get crude. Crude was purified by Combi Flash® using 5-6% ethyl acetate in hexanes to afford (6Z,9Z)-hexatriaconta-6,9-diene-18,19-diol (Int-5) as white semi solid.

Yield: 600 mg (46.5%); Characterized by ¹H-NMR

### Step-4: Synthesis of (2-{4-[(8Z,11Z)-heptadeca-8,11-dien-1-yl]-5-heptadecyl-1,3-dioxolan-2-yl}ethyl)dimethylamine (Lipid-4)

To a solution of Int-4 (4.5 g, 0.0084 mol) and Int-5 (488 mg, 0.00105 mol) in toluene (140 mL, 31 V), was added PTSA monohydride (2 g, 0.0105 mol) at 25-30° C. Resulting reaction mixture was refluxed at 130° C. for 18 h in a dean-stark apparatus. Progress of the reaction was monitored by TLC. After completion of reaction, reaction mixture was evaporated to remove toluene, residue obtained was treated with sodium bicarbonate solution and product was extracted with Ethyl acetate (3×100 mL). Organic layer was dried over Na₂SO₄ and concentrated under reduced pressure to get the crude. Crude was purified through combi flash, compound was eluted at 40-50% Ethyl acetate:Hexane. Pure fractions were evaporated to afford (2-{4-[(8Z,11Z)-heptadeca-8,11-dien-1-yl]-5-heptadecyl-1,3-dioxolan-2-yl}ethyl)dimethylaminc (Lipid-4) as brown liquid.

Yield: 2 g (38.4%); Characterized by 1H-NMR and LCMS

LCMS: (EI, m/z) calcd for C₄₁H₇₉NO₂ [M+H]: 618.8; HPLC Purity: 74.09%

¹H NMR (400 MHZ, CDCl₃): 5.40-5.32 (4H, m), 5.04-5.01 (1H, t, J=4.8 Hz), 3.56-3.55 (2H, m), 2.79-2.76 (2H, t, J=6.4 Hz), 2.58-2.40 (2H, m), 2.31 (6H, s), 2.08-2.03 (4H, q, J₁=13.6 Hz, J₂=13.6 Hz), 1.89-1.87 (2H, t, J=3.2 Hz), 1.54-1.53 (4H, m), 1.44-1.26 (44H, m), 0.91-0.86 (6H, m).

### Example 5: Synthesis of Lipid-5

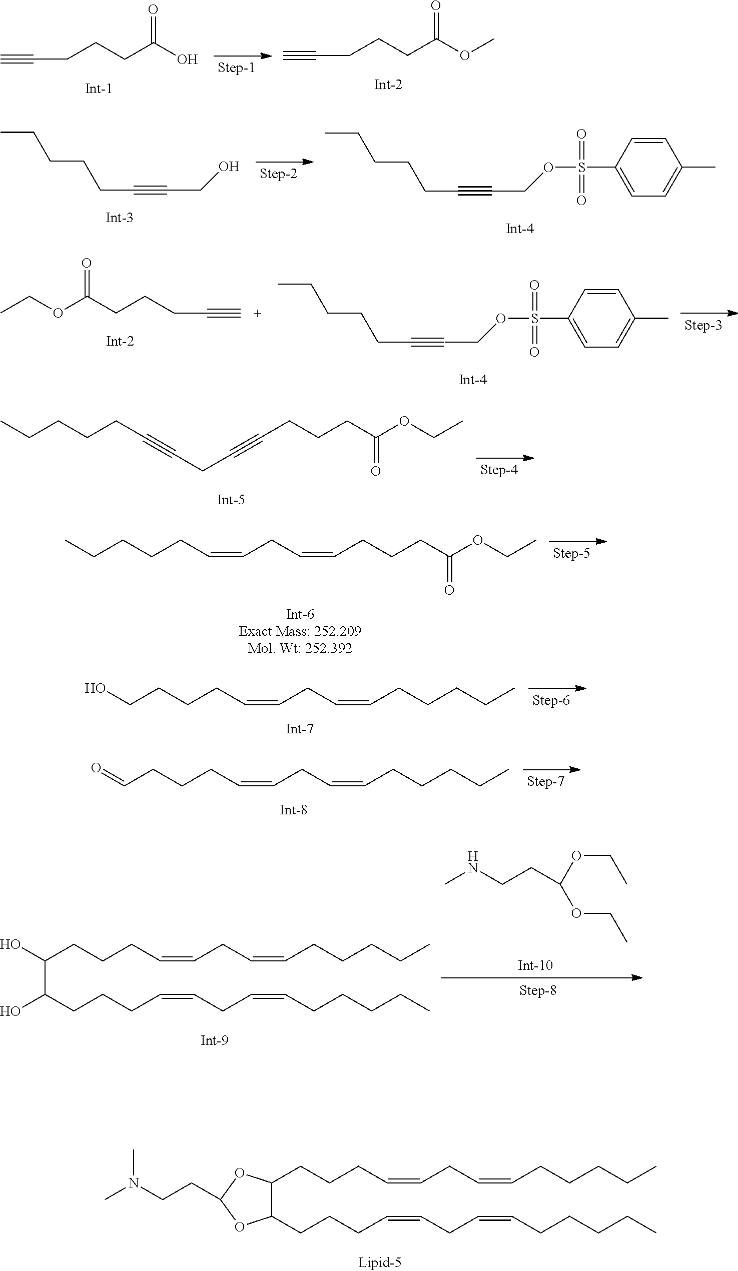

### Step-1: Synthesis of Hex-5-ynoic acid ethyl ester (Int-2)

To a stirred solution of Int-1 (20 g, 0.178 mol) in ethanol (50 mL, 2.5 V) was added Conc H₂SO₄ (0.1 V) slowly at 0-5° C. Resultant reaction mixture was refluxed at 80° C. for 18 h. Progress of the reaction was monitored by TLC. After 18 h, TLC shows completion of the reaction. Reaction mixture was cooled to 0-5° C. and pH was adjusted to 8 using 10% NaHCO₃ solution. Reaction mixture was evaporated to remove excess of ethanol and extracted with Ethyl acetate (2×200 mL). Combined organic layer was dried over sodium sulphate and concentrated at 45° C. to get crude Hex-5-ynoic acid ethyl ester (Int-2) as a pale brown liquid. Crude was taken as such for next reaction.

Yield: 20 g (crude); Characterized by 1H-NMR.

### Step-2: Synthesis of Toluene-4-sulfonic acid oct-2-ynyl ester (Int-4)

To a stirred solution of Int-3 (20 g, 0.15 mol) in Acetone (100 ml, 5 v) was added P-toluene sulfonyl chloride (42.2 g, 0.22 mol) at 0-5° C. To the above reaction mixture, a premixed solution of KOH (13.3 g, 0.23 mol) and K₂CO₃ (10.9 g, 0.079 mol) in water (100 mL, 5 V) was added slowly at 0-5° C. Resulting suspension was allowed to stir at 25-30° C. for 18 h. Progress of the reaction was monitored by TLC. After 18 h, TLC shows new non-polar spot formation. Reaction was quenched by adding 10% NaCl solution and extracted with DCM (3×100 mL). Combined organic layer was dried over Na₂SO₄ and concentrated at 45° C. to get the crude. Crude was purified through combi-flash, compound was eluted at 6-8% Ethyl acetate:Hexane. Pure fractions were evaporated to afford Toluene-4-sulfonic acid oct-2-ynyl ester (Int-4) as colourless liquid.

Yield: 15.8 g (36%); Characterized by 1H-NMR.

### Step-3: Synthesis of Tetradeca-5,8-diynoic acid ethyl ester (Int-5)

To a stirred suspension of sodium iodide (11.7 g, 0.078 mol) and copper iodide (14.96 g, 0.078 mol) in DMF (75 mL, 15 V) (pre-purged with argon) was added K₂CO₃ (9.87 g, 0.122 mol) at 25-30° C. Int-2 (5 g, 0.035 mol) followed by Int-4 (12 g, 0.042 mol) was added slowly at 25-30° C. Resulting pale yellowish suspension was stirred for 18 h at 25-30° C. Reaction was monitored by TLC. After 18 h, TLC shows complete consumption of Int-2. Reaction mixture was quenched by adding sat. NH₄Cl solution, filtered through celite bed. Filtrate was collected and extracted with MTBE (3×100 mL). Combined organic layer was dried over sodium sulphate and concentrated at 45° C. to get crude product. Crude was purified through combi-flash, compound was eluted at 6-8% Ethyl acetate:Hexane. Pure fractions were evaporated at 45° C. to afford Tetradeca-5,8-diynoic acid ethyl ester (Int-5) as brownish liquid.

Yield: 6.36 g (70.8%); Characterized by 1H-NMR

### Step-4: Synthesis of (5Z,8Z)-Tetradeca-5,8-dienoic acid ethyl ester (Int-6)

To an Ethanol (189 mL, 100 V) which is under continuous purging with H₂ gas was added Nickelacetatetetrahydrate (12.48 g, 0.05 mol) and cooled to 10° C. Sodium borohydride (1.9 g, 0.05) was added at 10-12° C. To the resulting black coloured reaction mixture Ethylene diamine (6 mL, 0.093 mol) followed by Int-5 (6.3 g, 0.022 mol) were added at 10° C. Reaction mixture was allowed to stir at 5-10° C. for 3 h with continuous purging of H₂ gas. Progress of the reaction was monitored by TLC (reaction need to be monitored for every half an hour). After 3 h, TLC shows completion of the reaction. Reaction was quenched by adding 1.5 M HCl and filtered through celite. Filtrate was collected and extracted with MTBE. Combined organic layer was dried over sodium sulphate and concentrated at 45° C. to get the crude. Crude was purified combi-flash, compound was eluted at 0-2% Ethyl acetate:Hexane. Pure fractions were evaporated to afford (5Z,8Z)-Tetradeca-5,8-dienoic acid ethyl ester (Int-6) brownish liquid.

Yield: 4.3 g (77%); Characterized by 1H-NMR

### Step-5: Synthesis of (5Z,8Z)-Tetradeca-5,8-dien-1-ol (Int-7)

To a stirred solution of Int-6 (4.3 g, 0.017 mol) in THF (43 mL, 10 V) was added LiAlH₄ (54.26 mL, 0.0085 mol, 2M in THF) at −25 to −30° C. over a period of 10 min. Reaction mixture was allowed to stir at same temperature for 1 h. Reaction was monitored by TLC. After 1 h, TLC shows completion of the reaction. Reaction mixture was quenched by adding saturated ammonium chloride solution at 0 to −5° C. and treated with ethyl acetate and stirred at 25-30° C. for 30 min. Reaction mixture was filtered through celite bed. Filtrate was washed with water and extracted with ethyl acetate. Combined organic layer was evaporated to get crude (5Z,8Z)-Tetradeca-5,8-dien-1-ol (Int-7) as pale yellow thick oil. Crude was as such taken for next step.

Yield: 3.18 g (crude); Characterized by ¹H-NMR

### Step-6: Synthesis of (5Z,8Z)-Tetradeca-5,8-dienal (Int-8)

To an ice cold solution of Int-7 (3.1 g, 0.014 mol) in DCM (62 mL, 20 V) was added dessmortin per iodinate (6.88 g, 0.016 mol) at 0-5° C. Reaction mixture was allowed to attain 25-30° C. and stirred at the same temperature for 1.5 h. Reaction was monitored by TLC. After 1.5 h, TLC shows completion of the reaction. Reaction mixture was quenched by adding 10% NaHCO₃ solution (80 ml, 16 V) at 0-5° C. and product was extracted with dichloromethane (3×50 mL). Combined organic layer was dried over Na₂SO₄ and concentrated at 45° C. to get crude. Crude was purified through combi-flash, compound was eluted at 3-4% ethyl acetate:hexane. Pure fractions were evaporated at 45° C. to afford (5Z,8Z)-Tetradeca-5,8-dienal (Int-8) as white solid.

Yield: 2.0 g (65.1%); Characterized by ¹H-NMR

### Step-7: Synthesis of (6Z,9Z,19Z,22Z)-Octacosa-6,9,19,22-tetraene-14,15-diol (Int-9)

To a stirred solution of Zinc dust (1.7 g, 0.026 mol) in 1,4-Dioxane (15 ml, 7.5 V) and DCM (15 ml, 7.5 V) at 5-10° C., Titanium chloride (2.1 ml, 0.019 mol) was added over a period of 10 min. Reaction mixture was allowed to stir at 5-10° C. for 30 min. A predissolved solution of Int-8 (2.0 g 0.0096 mol) in 1,4 dioxane (20 ml, 10 V) was added at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate extracted with ethyl acetate (3×100 mL). Combined organic layer was dried over anhydrous sodium sulphate and evaporated at 40° C. to get crude. Crude was purified by combi Flash® using 6-8% ethyl acetate in hexanes to afford (6Z,9Z,19Z,22Z)-Octacosa-6,9,19,22-tetraene-14,15-diol (Int-9) as white semi solid.

Yield: 0.83 g (41.5%)

### Step-8: Synthesis of {2-[4,5-Bis-((4Z,7Z)-trideca-4,7-dienyl)-[1,3]dioxolan-2-yl]-ethyl}-dimethyl-amine (Lipid-5)

To a solution of Int-9 (600 mg, 0.0014 mol) and Int-10 (342 mg, 0.0017 mol) in toluene (25 mL, 41 V), was added PTSA monohydride (342 mg, 0.0017 mol) at 25-30° C. Resulting reaction mixture was refluxed at 130° C. for 18 h in a dean-stark apparatus. Progress of the reaction was monitored by TLC, TLC shows completion of the reaction. The reaction mixture was evaporated to remove toluene, obtained residue was treated with sodium bicarbonate solution and product was extracted with Ethyl acetate (3×50 mL). Organic layer was dried over anhydrous sodium sulphate and evaporated at 40° C. to get crude. Crude was purified through combi flash, product was eluted at 40-50% Ethyl acetate:Hexane. Pure fractions were evaporated to afford {2-[4,5-Bis-((4Z,7Z)-trideca-4,7-dienyl)-[1,3]dioxolan-2-yl]-ethyl}-dimethyl-amine (Lipid-5) as brown liquid.

Yield: 438 mg (60.9%); Characterized by 1H-NMR and LCMS

LCMS: (EI, m/z) calcd for C₃₃H₅₉NO₂ [M+H]: 502.5; HPLC Purity: 76.16%

¹H NMR (400 MHZ, CDCl₃): 5.41-5.34 (8H, m), 5.02 (1H, m), 3.57-3.56 (2H, m), 2.79-2.76 (2H, t, J=6.0 Hz), 2.41-2.39 (2H, t, J=2.4 Hz), 2.24 (6H, s), 2.12-2.02 (8H, m), 1.84-1.82 (2H, m), 1.57-1.54 (4H, m), 1.43-1.32 (2H, m), 1.30-1.26 (16H, m), 0.89-0.88 (6H, t, J=4.0 Hz);

### Example 6: Synthesis of Lipid-6

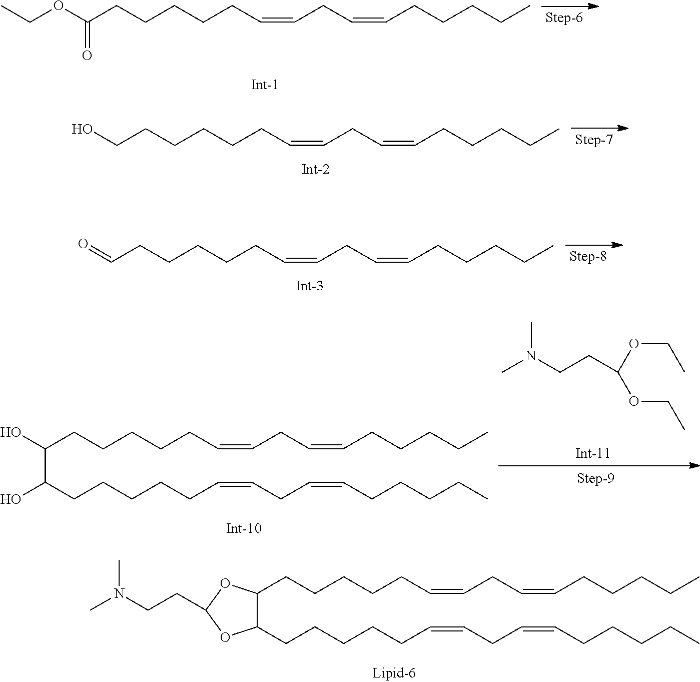

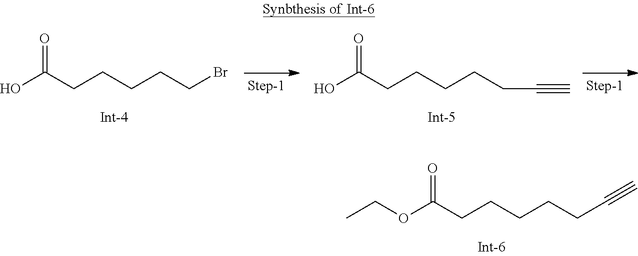

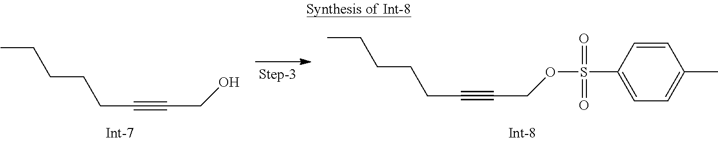

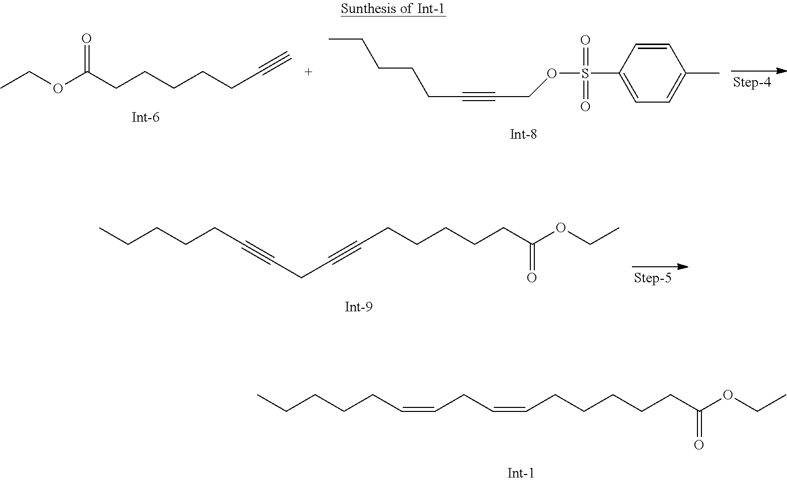

### Step-1: Synthesis of Oct-7-ynoic acid (Int-5)

To a stirred suspension of lithium acetylide ethylene diamine complex (23 g, 0.23 mol) in DMSO (50 mL, 5 V) was added a predissolved solution of Int-4 (15 g, 0.076 mol) in DMSO (75 mL, 5V) at 0-5° C. Resulting reaction mixture was stirred at 25-30° C. for 2.5 h. Progress of the reaction was monitored by TLC. After 2.5 h TLC shows completion of the reaction. Reaction was quenched by pouring it into ice cold brine solution, further acidified to PH˜ 4-5 using 1.5 M HCl and product was extracted with DCM (3×150 mL). Combined organic layer was dried over sodium sulphate and evaporated at 45° C. under reduced pressure to get crude Oct-7-ynoic acid (Int-5) as brown liquid. Crude was taken as such for next reaction.

Yield: 18.1 g (crude); Characterized by 1H-NMR

### Step-2: Synthesis of Ethyl oct-7-ynoate (Int-6)

To a stirred solution of Int-5 (18 g, 0.12 mol) in ethanol (90 mL, 5 V) was added Conc H₂SO₄ (0.1 V) slowly at 0-5° C. Resultant reaction mixture was refluxed at 80° C. for 18 h. Progress of the reaction was monitored by TLC. After 18 h, TLC shows completion of the reaction. Reaction mixture was cooled to 0-5° C. and pH was adjusted to 8 using 10% NaHCO₃ solution. Reaction mixture was evaporated to remove excess of ethanol and extracted with Ethyl acetate (2×75 mL). Combined organic layer was dried over sodium sulphate and concentrated at 45° C. to get crude Ethyl oct-7-ynoate (Int-6) as a pale brown liquid. Crude was taken as such for next reaction.

Yield: 10.38 g (crude); Characterized by 1H-NMR

### Step-3: Synthesis of Oct-2-yn-1-yl 4-methylbenzene-1-sulfonate (Int-8)

To a stirred solution of Int-7 (15 g, 0.118 mol) in Acetone (75 ml, 5 v) was added P-toluene sulfonyl chloride (31.7 g, 0.116 mol) at 0-5° C. To the above reaction mixture, a premixed solution of KOH (10 g, 0.178 mol) and K₂CO₃ (8.2 g, 0.059 mol) in water (75 mL, 5 V) was added slowly at 0-5° C. Resulting suspension was allowed to stir at 25-30° C. for 18 h. Progress of the reaction was monitored by TLC. After 18 h, TLC shows new non-polar spot formation. Reaction was quenched by adding 10% NaCl solution and extracted with DCM (3×100 mL). Combined organic layer was dried over Na₂SO₄ and concentrated at 45° C. to get the crude. Crude was purified through combi-flash, compound was eluted at 6-8% Ethyl acetate:Hexane. Pure fractions were evaporated to afford Oct-2-yn-1-yl 4-methylbenzene-1-sulfonate (Int-8) as colourless liquid.

Yield: 15 g (60.9%); Characterized by 1H-NMR

### Step-4: Synthesis of Ethyl tetradeca-5,8-diynoate (Int-9)

To a stirred suspension of sodium iodide (20.6 g, 0.13 mol) and copper iodide (25.19 g, 0.13 mol) in DMF (155 mL, 15 V) (pre-purged with argon) was added K₂CO₃ (16.9 g, 0.122 mol) at 25-30° C. Int-6 (10.3 g, 0.061 mol) followed by Int-8 (20.6 g, 0.073 mol) was added slowly at 25-30° C. Resulting pale yellowish suspension was stirred for 18 h at 25-30° C. Reaction was monitored by TLC. After 18 h, TLC shows complete consumption of Int-6. Reaction mixture was quenched by adding sat·NH₄Cl solution, filtered through celite bed. Filtrate was collected and extracted with MTBE (3×100 mL). Combined organic layer was dried over sodium sulphate and concentrated at 45° C. to get crude product. Crude was purified through combi-flash, compound was eluted at 6-8% Ethyl acetate:Hexane. Pure fractions were evaporated at 45° C. to afford Ethyl tetradeca-5,8-diynoate (Int-9) as brownish liquid. Yield: 6.1 g (impure); Characterized by 1H-NMR

### Step-5: Synthesis of Ethyl (7Z,10Z)-hexadeca-7,10-dienoate (Int-1)

To an Ethanol (300 mL, 100 V) which is under continuous purging with H₂ gas was added Nickelacetatetetrahydrate (5.4 g, 0.021 mol) and cooled to 10° C. Sodium borohydride (1.64 g, 0.043) was added at 10-12° C. To the resulting black coloured reaction mixture Ethylene diamine (6 g, 0.093 mol) followed by Int-9 (6 g, 0.217 mol) were added at 10° C. Reaction mixture was allowed to stir at 5-10° C. for 3 h with continuous purging of H₂ gas. Progress of the reaction was monitored by TLC (reaction need to be monitored for every half an hour). After 3 h, TLC shows completion of the reaction. Reaction was quenched by adding 1.5 M HCl and filtered through celite. Filtrate was collected and extracted with MTBE. Combined organic layer was dried over sodium sulphate and concentrated at 45° C. to get the crude. Crude was purified combi-flash, compound was eluted at 0-2% Ethyl acetate:Hexane. Pure fractions were evaporated to afford Ethyl (7Z,10Z)-hexadeca-7,10-dienoate (Int-1) brownish liquid. Yield: 1.4 g (46%); Characterized by 1H-NMR

### Step-6: Synthesis of (7Z,10Z)-Hexadeca-7,10-dien-1-ol (Int-2)

To a stirred solution of Int-1 (3 g, 0104 mol) in THF (30 mL, 10 V) was added LiAlH₄ (5.2 mL, 0.0104 mol, 2M in THF) at −25 to −30° C. over a period of 10 min. Reaction mixture was allowed to stir at same temperature for 1 h. Reaction was monitored by TLC. After 1 h, TLC shows completion of the reaction. Reaction mixture was quenched by adding saturated ammonium chloride solution at 0 to −5° C. and treated with ethyl acetate and stirred at 25-30° C. for 30 min. Reaction mixture was filtered through celite bed. Filtrate was washed with water and extracted with ethyl acetate. Combined organic layer was dried over sodium sulphate and concentrated at 45° C. to get crude (7Z,10Z)-Hexadeca-7,10-dien-1-ol (Int-2) as pale yellow thick oil. Crude as such taken for next step.

Yield: 2.4 g (crude); Characterized by 1H-NMR

### Step-7: Synthesis of Octadecanal (Int-4)

To an ice cold solution of Int-3 (2.4 g, 0.010 mol) in DCM (48 mL, 20 V) was added dessmortin per iodinate (4.69 g, 0.011 mol) at 0-5° C. Reaction mixture was allowed to attain 25-30° C. and stirred at the same temperature for 1.5 h. Reaction was monitored by TLC. After 1.5 h, TLC shows completion of the reaction. Reaction mixture was quenched by adding 10% NaHCO₃ solution (40 ml, 16 V) at 0-5° C. and product was extracted with dichloromethane (3×40 mL). Combined organic layer was dried over Na₂SO₄ and concentrated at 45° C. to get crude. Crude was purified through combi-flash, compound was eluted at 3-4% ethyl acetate:hexane. Pure fractions were evaporated at 45° C. to afford Octadecanal (Int-4) as white solid. Yield: 1.9 g (80.16%); Characterized by 1H-NMR

### Step-8: Synthesis of (6Z,9Z,23Z,26Z)-dotriaconta-6,9,23,26-tetraene-16,17-diol (Int-10)

To a stirred solution of Zinc dust (1.43 g, 0.022 mol) in 1,4-Dioxane (14.3 ml, 15 V) and DCM (14.3 ml, 15 V) at 5-10° C., Titanium chloride (1.78 ml, 0.016 mol) was added over a period of 10 min. Reaction mixture was allowed to stir at 5-10° C. for 30 min. A predissolved solution of Int-3 (1.9 g 0.008 mol) in 1,4 dioxane (38 ml, 20 V) was added at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate extracted with ethyl acetate (3×60 mL). Combined organic layer was dried over anhydrous sodium sulphate and evaporated at 40° C. to get crude. Crude was purified by combi Flash® using 6-8% ethyl acetate in hexanes to afford (6Z,9Z,23Z,26Z)-dotriaconta-6,9,23,26-tetraene-16,17-diol (Int-10) as white semi solid.

Yield: 1.1 g (55%)

### Step-9: Synthesis of (2-{4,5-Bis [(6Z,9Z)-pentadeca-6,9-dien-1-yl]-1,3-dioxolan-2-ylethyl)dimethylamine (Lipid-6)

To a solution of Int-10 (600 mg, 0.0012 mol) and Int-11 (275 mg, 0.0015 mol) in toluene (25 mL, 41 V), was added PTSA monohydride (286.5 mg, 0.0015 mol) at 25-30° C. Resulting reaction mixture was refluxed at 130° C. for 18 h in a dean-stark apparatus. Progress of the reaction was monitored by TLC, TLC shows completion of the reaction. The reaction mixture was evaporated to remove toluene, obtained residue was treated with sodium bicarbonate solution and product was extracted with Ethyl acetate (3×100 mL). Organic layer was dried over anhydrous sodium sulphate and evaporated at 40° C. to get crude. Crude was purified through combi flash, product was eluted at 40-50% Ethyl acetate:Hexane. Pure fractions were evaporated to afford (2-{4,5-Bis [(6Z,9Z)-pentadeca-6,9-dien-1-yl]-1,3-dioxolan-2-yl}ethyl)dimethylamine (Lipid-6) as pale brown liquid.

Yield: 245 mg (35%); Characterized by ¹H-NMR and LCMS

LCMS: (EI, m/z) calcd for C₃₇H₆₇NO₂ [M+H]: 558.7; HPLC Purity: 63.42%

¹H NMR (400 MHZ, CDCl₃): 5.39-5.38 (8H, m), 5.01 (1H, m), 3.56-3.55 (2H, m), 2.79-2.76 (2H, t, J=6.8 Hz), 2.42-2.40 (2H, t, J=2.4 Hz), 2.24 (6H, s), 2.07-2.03 (8H, m), 1.84-1.82 (2H, t, J=9.2 Hz), 1.48-1.40 (6H, m), 1.30-1.27 (24H, m), 0.89-0.88 (6H, t, J=4.0 Hz)

### Example 7: Synthesis of Lipid-7

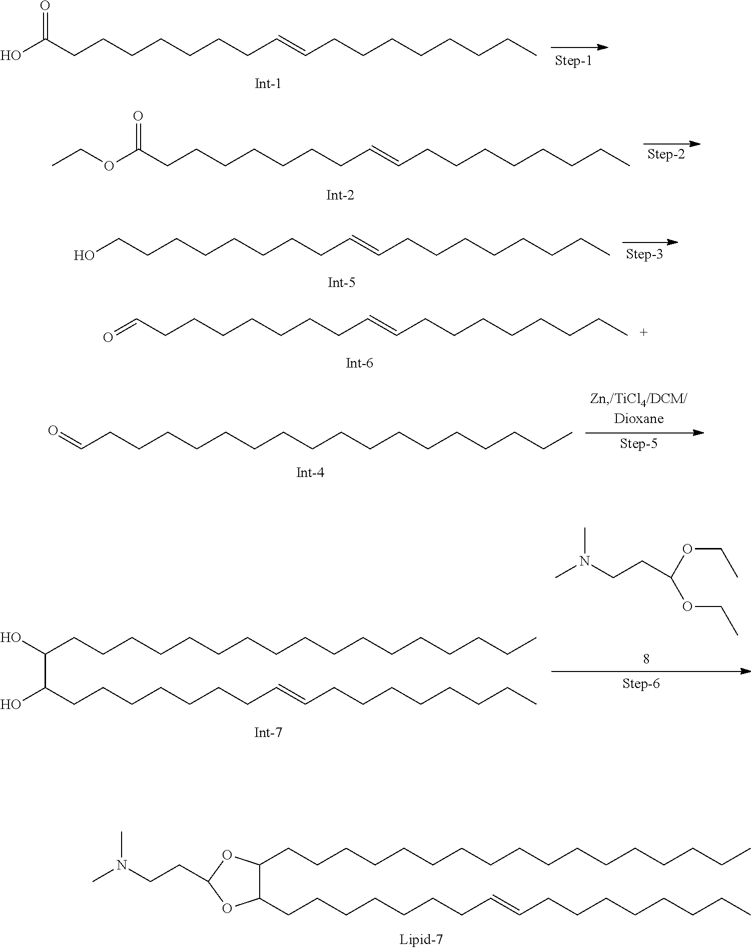

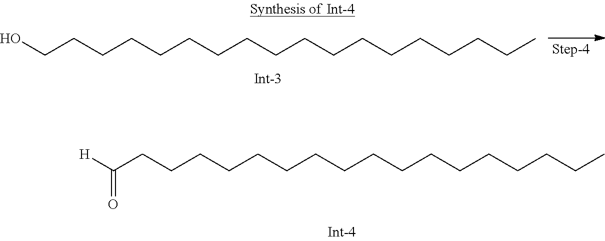 [SMILES: CCCCCCCCCCCCCCCCCCO ; CCCCCCCCCCCCCCCCCC=O]

### Step-1: Synthesis of Ethyl (9E)-Octadec-9-enoate (Int-2)

Conc H₂SO₄ (0.5 mL, 0.1 V) was added dropwise to cold ethanol (50 mL, 10 V) at 0-5° C. A premixed solution of Int-1 (5.0 g, 0.017 mol) in ethanol (25 mL, 5 V) was added drop wise at 0-5° C. Resulting reaction mixture was refluxed at 80° C. for 18 h. Progress of the reaction was monitored by TLC, TLC of the reaction shows completion of the reaction. Reaction mixture was cooled to 0-5° C. and pH was adjusted to 8 using 10% NaHCO₃ solution. Reaction mixture was evaporated to remove excess of ethanol and extracted with dichloromethane (2×50 mL), organic layer was washed with water and dried over anhydrous sodium sulphate and evaporated at 40° C. to afford crude Ethyl (9E)-Octadec-9-enoate (Int-2) as pale brown liquid. Crude was used as such for the next reaction.

Yield: 5.0 g (crude)

### Step-2: Synthesis of (9E)-octadec-9-en-1-ol (Int-5)

LiAlH₄ (8 mL, 0.032 mol, 2M in THF) was added slowly to a solution of Int-2 (5 g, 0.016 mol) in THF (30 mL, 10 V) at −25 to −30° C. over a period of 5 min. Resulting reaction mixture was allowed to stir at −25 to −30° C. for 1 h. Progress of the reaction was monitored by TLC. Reaction mixture was quenched by adding saturated ammonium chloride solution (60 mL) at 0-5° C., diluted with ethyl acetate (200 mL) and stirred at 25-30° C. for 30 min. Reaction mixture was filtered through celite bed. Filtrate was washed with water and extracted with ethyl acetate (3×50 mL). Combined organic layer was dried over anhydrous sodium sulphate and evaporated at 40° C. to afford crude (9E)-octadec-9-en-1-ol (Int-5) as pale brown liquid. Crude was used as such for next reaction.

Yield: 3.92 g (crude)

### Step-3: Synthesis of (9E)-octadec-9-enal (Int-6)

To an ice cold solution of Int-5 (3.9 g, 0.014 mol) in DCM (78 mL, 20 V) was added Dess-Martin periodinane (6.78 g, 0.015 mol) at 0-5° C. Reaction was allowed to stir at 25-30° C. for 1.5 h. Progress of the reaction was monitored by TLC. After 1.5 h TLC shows complete consumption of Int-3. Reaction mixture was cooled to 0-5° C. and quenched by adding saturated sodium bicarbonate solution (25 mL, 10 V) at 0-5° C. and product was extracted with DCM (3×60 mL). Combined organic layer was dried over anhydrous sodium sulphate and evaporated at 40° C. to get crude. Crude was purified by combi Flash® using 3 to 4% EtOAc in hexanes to afford (9E)-octadec-9-enal (Int-6) as colourless liquid.

Yield: 3.0 g (77%)

### Step-4: Synthesis of Octadecanal (Int-4)

To an ice cold solution of Int-3 (5 g, 0.018 mol) in DCM (100 mL, 20 V) was added Dess-Martin periodinane (8.64 g, 0.020 mol) at 0-5° C. Reaction was allowed to stir at 25-30° C. for 1.5 h. Progress of the reaction was monitored by TLC. After 1.5 h TLC shows complete consumption of Int-3. Reaction mixture was cooled to 0-5° C. and quenched by adding saturated sodium bicarbonate solution (30 mL, 10 V) at 0-5° C. and product was extracted with DCM (3×50 mL). Combined organic layer was dried over anhydrous sodium sulphate and evaporated at 40° C. to get crude. Crude was purified by combi Flash® using 3 to 4% EtOAc in hexanes to afford Octadecanal (Int-6) as colourless liquid.

Yield: 4.2 g (82%)

### Step-5: Synthesis of (9E)-hexatriacont-9-ene-18,19-diol (Int-7)

To a stirred solution of Zinc dust (3.34 g, 0.051 mol) in 1,4-Dioxane (37.5 mL, 15 V) and DCM (37.5 mL, 15 V) at 5-10° C., Titanium chloride (4.15 ml, 0.037 mol) was added over a period of 10 min. Reaction mixture was allowed to stir at 5-10° C. for 30 min. A predissolved solution of Int-4 (2.5 g, 0.093 mol) and Int-6 (2.48 g, 0.093 mol) in 1,4 dioxane (50 mL, 20 V) was added to reaction mixture at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate was extracted with ethyl acetate (3×60 mL). Combined organic layer was dried over anhydrous sodium sulphate and evaporated at 40° C. to get crude. Crude was purified by combi Flash® using 6-8% ethyl acetate in hexanes to afford (9E)-hexatriacont-9-ene-18,19-diol (Int-7) as white semi solid.

Yield: 1.3 g (26%)

### Step-6: Synthesis of (2-{4-[(8E)-heptadec-8-en-1-yl]-5-heptadecyl-1,3-dioxolan-2-yl}ethyl)dimethylamine (Lipid-7)

To a solution of Int-7 (1.3 g, 0.0024 mol and Int-8 (530 mg, 0.003 mol) in toluene (50 mL, 38 V), was added PTSA monohydride (693 mg, 0.0036 mol) at 25-30° C. Resulting reaction mixture was refluxed at 130° C. for 18 h in a dean-stark apparatus. Progress of the reaction was monitored by TLC, TLC shows completion of the reaction. The reaction mixture was evaporated to remove toluene, obtained residue was treated with sodium bicarbonate solution (20 mL) and product was extracted with Ethyl acetate (3×40 mL). Organic layer was dried over anhydrous sodium sulphate and evaporated at 40° C. to get crude. Crude was purified through combi flash, product was eluted at 25-45% Ethyl acetate:Hexane. Pure fractions were evaporated to afford (2-{4-[(8E)-heptadec-8-en-1-yl]-5-heptadecyl-1,3-dioxolan-2-yl}ethyl)dimethylamine (Lipid-7) as pale brown liquid.

Yield: 800 mg (55%); Characterized by 1H-NMR and LCMS

LCMS: (EI, m/z) calcd for C₄₁H₈₁NO₂ [M+H]: 620.8; HPLC Purity: 55.29%

¹H NMR (400 MHZ, CDCl₃): 5.39-5.38 (2H, t, J=3.6 Hz), 5.03-5.00 (1H, t, J=4.8 Hz), 3.56-3.55 (2H, m), 2.44-2.39 (2H, m), 2.24 (6H, s), 1.97-1.94 (5H, t, J=4.8 Hz), 1.85-1.75 (3H, m), 1.62-1.60 (6H, m), 1.34-1.18 (48H, m), 0.90-0.88 (6H, m)

### Example 8: Synthesis of Lipid-8

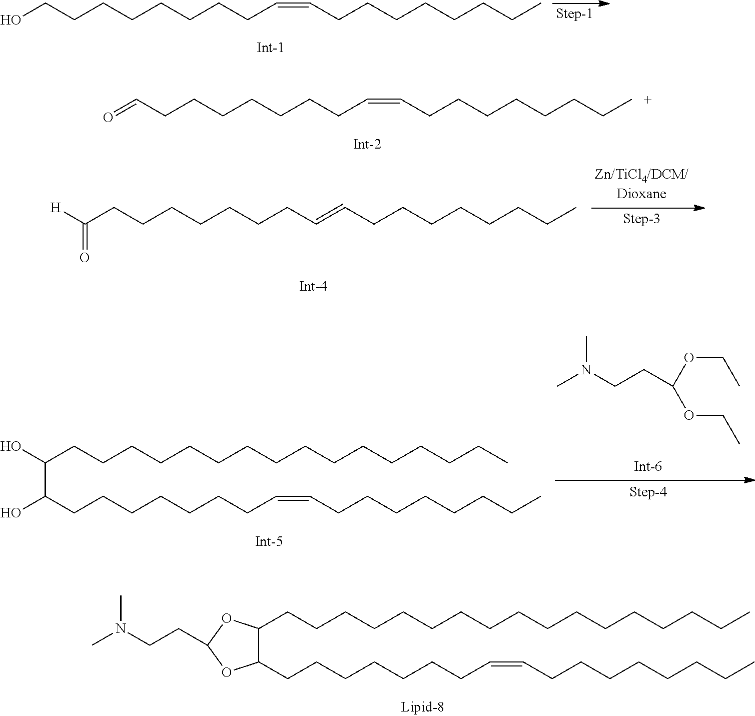

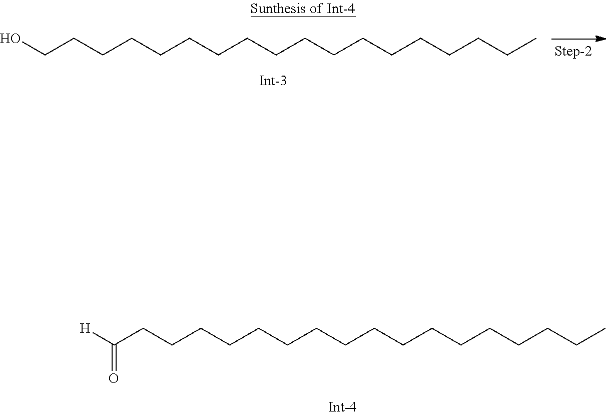 [SMILES: CCCCCCCCCCCCCCCCCCO ; CCCCCCCCCCCCCCCCCC=O]

### Step-1: Synthesis of (9Z)-Octadec-9-enal (Int-2)

To an ice cold solution of Int-1 (5 g, 0.018 mol) in DCM (100 mL, 20 V), was added Dess-Martin periodinane (8.68 g, 0.020 mol) at 0-5° C. Reaction was allowed to stir at 25-30° C. for 1.5 h. Progress of the reaction was monitored by TLC. After 1.5 h TLC shows complete consumption of Int-1. Reaction mixture was cooled to 0-5° C. and quenched by adding saturated sodium bicarbonate solution (50 mL, 10 V) at 0-5° C. and product was extracted with DCM (3×100 mL). Combined organic layer was dried over anhydrous sodium sulphate and evaporated at 40° C. to get crude. Crude was purified by combi Flash® using 3 to 4% EtOAc in hexanes to afford (9Z)-Octadec-9-enal (Int-2) as colourless liquid.

Yield: 3.5 g (70.5%)

### Step-2: Synthesis of Octadecanal (Int-4)

To an ice cold solution of Int-3 (15 g, 0.0554 mol), in DCM (300 mL, 20 V) was added Dess-Martin periodinane (28.3 g, 0.066 mol) at 0-5° C. Reaction was allowed to stir at 25-30° C. for 1.5 h. Progress of the reaction was monitored by TLC. After 1.5 h TLC shows complete consumption of Int-3. Reaction mixture was cooled to 0-5° C. and quenched by adding saturated sodium bicarbonate solution (150 mL, 10 V) at 0-5° C. and product was extracted with DCM (3×50 mL). Combined organic layer was dried over anhydrous sodium sulphate and evaporated at 40° C. to get crude. Crude was purified by combi Flash® using 3 to 4% EtOAc in hexanes to afford Octadecanal (Int-4) as white solid.

Yield: 11.6 g (77.9%)

### Step-3: Synthesis of (9Z)-Hexatriacont-9-ene-18,19-diol (Int-5)

To a stirred solution of Zinc dust (4.68 g, 0.071 mol) in 1,4-Dioxane (52 ml, 15 V) and DCM (52 ml, 15 V) at 5-10° C., Titanium chloride (5.8 ml, 0.052 mol) was added over a period of 10 min. Reaction mixture was allowed to stir at 5-10° C. for 30 min. A predissolved solution of Int-2 (3.5 g 0.013 mol) and Int-4 (3.47 g, 0.013 mol) in 1,4 dioxane (70 ml, 20 V) was added at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate extracted with ethyl acetate (3×20 mL). Combined organic layer was dried over anhydrous sodium sulphate and evaporated at 40° C. to get crude. Crude was purified by combi Flash® using 5-6% ethyl acetate in hexanes to afford (9Z)-Hexatriacont-9-ene-18, 19-diol (Int-5) as white semi solid.

Yield: 3 g (42.8%); Characterized by ¹H-NMR

### Step-4: Synthesis of (2-{4-[(8Z)-Heptadec-8-en-1-yl]-5-heptadecyl-1,3-dioxolan-2-yl}ethyl)dimethylamine (Lipid-8)

To a stirred solution of Int-5 (1.4 g, 0.0026 mol) and Int-6 (574 mg, 0.0032 mol) in toluene (50 mL, 35 V), was added PTSA monohydride (623 mg, 0.0032 mol) at 25-30° C. Reaction mixture was refluxed at 130° C. for 24 h in a dean-stark apparatus. Progress of the reaction was monitored by TLC, after completion of reaction, reaction mixture was evaporated at 40° C. to remove toluene. Obtained residue was treated with sodium bicarbonate solution and product was extracted with ethyl acetate (3×100 mL). Combined organic layer was dried over anhydrous sodium sulphate and evaporated at 40° C. to get crude. Crude was purified by combi Flash® using 25 to 45% EtOAc in hexanes to afford Lipid-8 (600 mg, 37.5%) as brown liquid. Yield: 600 mg (37.5%); Characterized by 1H-NMR and LCMS

LCMS: (EI, m/z) calcd for C₄₁H₈₁NO₂ [M+H]: 620.8; HPLC Purity: 61.38%

¹H NMR (400 MHZ, CDCl₃): 5.36-5.34 (2H, m), 5.01 (1H, m), 3.56-3.55 (2H, m), 2.45-2.41 (2H, q, J₁=8.8 Hz, J₂=8.8 Hz), 2.25 (6H, s), 2.02-1.99 (4H, t, J=6.0 Hz), 1.85-1.81 (2H, m), 1.56-1.40 (6H, m), 1.44-1.26 (50H, m), 0.91-0.86 (6H, m)

### Example 9: Synthesis of Lipid-9

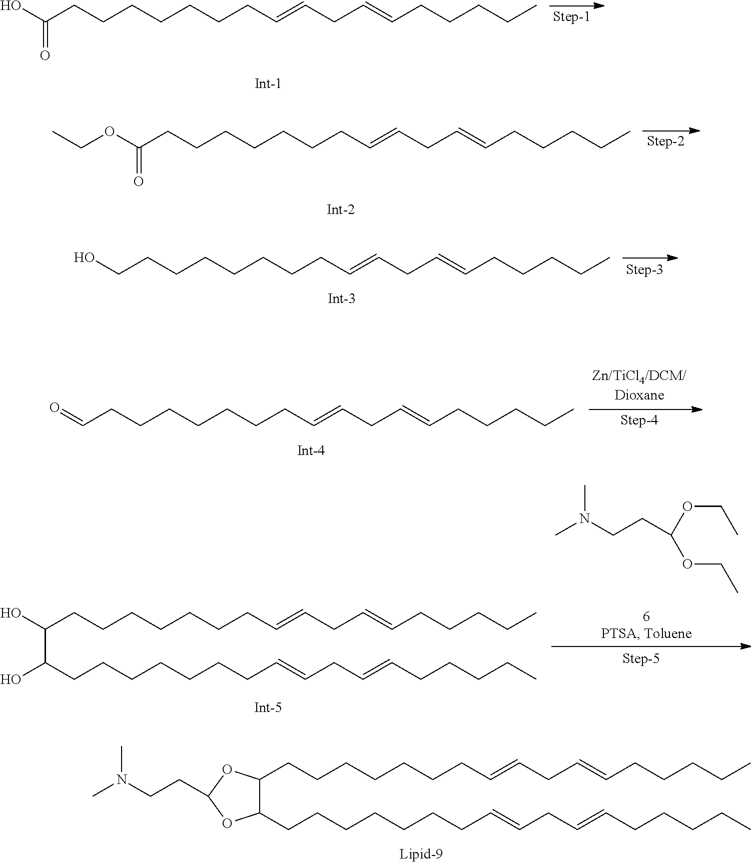

### Step-1: Synthesis of Ethyl (9E,12E)-octadeca-9,12-dienoate (Int-2)

Conc H₂SO₄ (0.5 mL, 0.1 V) was added dropwise to cold ethanol (15 mL, 5 V) at 0-5° C. A premixed solution of Int-1 (3.0 g, 0.01 mol) in ethanol (15 mL, 5 V) was added drop wise at 0-5° C. Resulting reaction mixture was refluxed at 80° C. for 18 h. Progress of the reaction was monitored by TLC, TLC of the reaction shows completion of the reaction. Reaction mixture was cooled to 0-5° C. and pH was adjusted to 8 using 10% NaHCO₃ solution. Reaction mixture was evaporated to remove excess of ethanol and extracted with dichloromethane (2×50 mL), organic layer was washed with water and dried over anhydrous sodium sulphate and evaporated at 40° C. to afford crude Ethyl (9E,12E)-octadeca-9,12-dienoate (Int-2) (3.0 g) as a pale brown liquid. Crude was used as such for the next reaction.

Yield: 3.0 g (crude)

### Step-2: Synthesis of (9E,12E)-octadeca-9,12-dien-1-ol (Int-3)

LiAlH₄ (4.8 mL, 0.0026, 2M in THF) was added slowly to a solution of Int-2 (3 g, 0.0026 mol) in THF (30 mL, 10 V) at −25 to −30° C. over the period of 5 min. Resulting reaction mixture was allowed to stir at −25 to −30° C. Progress of the reaction was monitored by TLC. Reaction mixture was quenched by adding saturated ammonium chloride solution (100 mL) at 0-5° C., diluted with ethyl acetate (100 mL) and stirred at 25-30° C. for 30 min. Reaction mixture was filtered through celite bed. Filtrate was washed with water and extracted with ethyl acetate (3×50 mL). Combined organic layer was dried over anhydrous sodium sulphate and evaporated at 40° C. to afford crude (9E,12E)-octadeca-9,12-dien-1-ol (Int-3) as pale yellow thick oil. Crude was used as such for next reaction.

Yield: 2.32 g (crude)

### Step-3: Synthesis of (9E,12E)-octadeca-9,12-dienal (Int-4)

To an ice cold solution of Int-3 (2.25 g, 0.0084 mol) in DCM (45 mL, 20 V) was added Dess-Martin periodinane (4.3 g, 0.010 mol) at 0-5° C. Reaction was allowed to stir at 25-30° C. for 1.5 h. Progress of the reaction was monitored by TLC. After 1.5 h TLC shows complete consumption of Int-3. Reaction mixture was cooled to 0-5° C. and quenched by adding saturated sodium bicarbonate solution (25 mL, 11 V) at 0-5° C. and product was extracted with DCM (3×30 mL). Combined organic layer was dried over anhydrous sodium sulphate and evaporated at 40° C. to get crude. Crude was purified by combi Flash® using 3 to 4% EtOAc in hexanes to afford (9E,12E)-octadeca-9,12-dienal (Int-4) as colourless liquid.

Yield: 1.5 g (67%)

### Step-4: Synthesis of (6E,9E,27E,30E)-Hexatriaconta-6,9,27,30-tetraene-18,19-diol (Int-5)

To a stirred solution of Zinc dust (1.01 g, 0.015 mol) in 1,4-Dioxane (11.25 mL, 15 V) and DCM (11.25 mL, 15 V) at 5-10° C., Titanium chloride (1.24 ml, 0.052 mol) was added over a period of 10 min. Reaction mixture was allowed to stir at 5-10° C. for 30 min. A predissolved solution of Int-4 (0.75 g, 0.013 mol) in 1,4 dioxane (70. mL, 20 V) was added to reaction mixture at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate extracted with ethyl acetate (3×20 mL). Combined organic layer was dried over anhydrous sodium sulphate and evaporated at 40° C. to get crude. Crude was purified by combi Flash® using 5-6% ethyl acetate in hexanes to afford (6E,9E,27E,30E)-Hexatriaconta-6,9,27,30-tetraene-18,19-diol (Int-5) as white semi solid.

Yield: 690 mg (46%)

### Step-5: Synthesis of (2-{4,5-Bis [(8E,11E)-heptadeca-8,11-dien-1-yl]-1,3-dioxolan-2-yl}ethyl)dimethylamine (Lipid-9)

To a solution of Int-5 (670 mg, 0.0012 mol) and Int-6 (276 mg, 0.0015 mol) in toluene (25 mL, 37 V), was added PTSA monohydride (361 mg, 0.0018 mol) at 25-30° C. Resulting reaction mixture was refluxed at 130° C. for 18 h in a dean-stark apparatus. Progress of the reaction was monitored by TLC, TLC shows completion of the reaction. The reaction mixture was evaporated to remove toluene, obtained residue was treated with sodium bicarbonate solution and product was extracted with Ethyl acetate (3×50 mL). Organic layer was dried over anhydrous sodium sulphate and evaporated at 40° C. to get crude. Crude was purified through combi flash, product was eluted at 25-45% Ethyl acetate:Hexane. Pure fractions were evaporated to afford (2-{4,5-Bis [(8E,11E)-heptadeca-8,11-dien-1-yl]-1,3-dioxolan-2-yl}ethyl)dimethylamine (Lipid-9) as pale brown liquid.

Yield: 454 mg (58%); Characterized by 1H-NMR and LCMS

LCMS: (EI, m/z) calcd for C₄₁H₇₅NO₂ [M+H]: 614.9; HPLC Purity: 98.2%

¹H NMR (400 MHZ, CDCl₃): 5.42-5.37 (8H, m), 5.03-5.00 (1H, m), 3.56-3.55 (2H, m), 2.67-2.66 (4H, m), 2.46-2.42 (2H, m), 2.26 (6H, s), 2.02-1.96 (8H, m), 1.88-1.82 (2H, m), 1.54-1.45 (6H, m), 1.39-1.24 (30H, m), 0.91-0.87 (6H, t, J=6.8 Hz)

### Example 10: Synthesis of Lipid-10

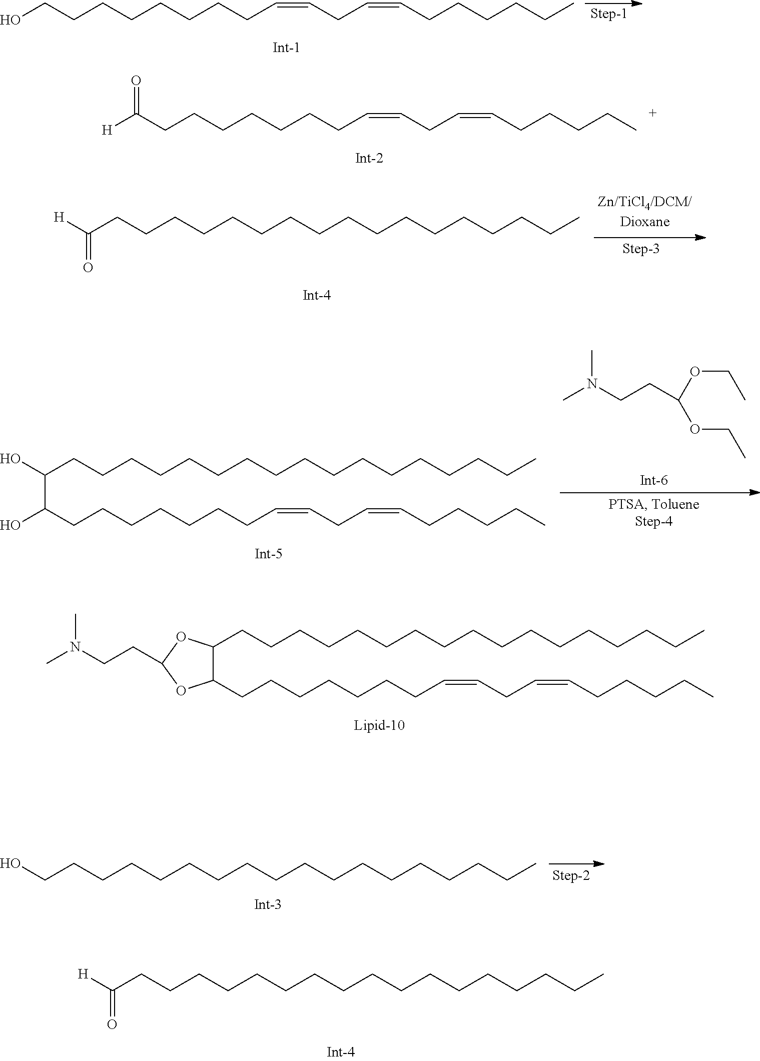

To an ice cold solution of Int-1 (10 g, 0.037 mol) in DCM (200 mL, 20 V) was added dessmortin periodinate (17.50 g, 0.041 mol) at 0-5° C. Resulting reaction mixture was allowed to attain 25-30° C. and stirred at same temperature for 1.5 h. Progress of the reaction was monitored by TLC, after 1.5 h TLC shows completion of reaction. Reaction mixture was quenched by adding 10% NaHCO₃ solution (100 mL, 10 V) at 0-5° C. and product was extracted with DCM (3×100 mL). Organic layer was dried over Na₂SO₄ and concentrated under reduced pressure to afford crude. Crude was purified through combi flash, compound was eluted at 3-4% ethyl acetate:hexane. Pure fractions were evaporated to get Ethyl (9Z,12Z)-octadeca-9,12-dienal (Int-2) as colourless liquid.

Yield: 6.8 g (68.8%)

### Step-2: Synthesis of Octadecanal (Int-4)

To an ice cold solution of Int-3 (15 g, 0.0554 mol) in DCM (300 mL, 20 V) was added dessmortin per iodinate (28.3 g, 0.066 mol) at 0-5° C. Reaction mixture was allowed to attain 25-30° C. and stirred at the same temperature for 1.5 h. Progress of the reaction was monitored by TLC, after 1.5 h TLC shows completion of the reaction. Reaction mixture was quenched by adding 10% NaHCO₃ solution (150 ml, 10 V) at 0-5° C. and product was extracted with DCM (3×150 mL). Organic layer was dried over Na₂SO₄ and concentrated under reduced pressure at 45° C. to get crude. Crude was purified through combi flash, compound was eluted at 3-4% ethyl acetate:hexane. Pure fractions were evaporated to afford Octadecanal (Int-4) as white solid.

Yield: 11.6 g (77.9%)

### Step-3: Synthesis of (6Z,9Z)-hexatriaconta-6,9-diene-18,19-diol (Int-5)

To a stirred solution of Zinc dust (10.8 g, 0.166 mol) in 1,4-Dioxane (120 mL, 15 V) and DCM (120 mL, 15 V) at 5-10° C., Titanium chloride (132.5 mL, 0.121 mol) was added over a period of 10 min. Reaction mixture was allowed to stir at 5-10° C. for 30 min. A predissolved solution of Int-2 (8 g, 0.030 mol) and Int-4 (8.1 g, 0.030 mol) in 1,4 dioxane (120 mL, 15 V) was added to reaction mixture at 5-10° C. and reaction mixture was allowed to attain 25-30° C. and stirred at same temperature for 3 h. Progress of the reaction was monitored by TLC. After completion of reaction, reaction mixture was quenched by adding 10% potassium carbonate solution at 0-5° C. and filtered through a plug of celite bed, filtrate was extracted with ethyl acetate (3×20 mL). Combined organic layer was dried over anhydrous sodium sulphate and evaporated at 40° C. to get crude. Crude was purified by Combi Flash® using 5-6% ethyl acetate in hexanes to afford (6Z,9Z)-hexatriaconta-6,9-diene-18,19-diol (Int-5) as white semi solid. Yield: 600 mg (46.5%)

### Step-4: Synthesis of (2-{4-[(8Z,11Z)-heptadeca-8,11-dien-1-yl]-5-heptadecyl-1,3-dioxolan-2-yl}ethyl)dimethylamine (Lipid-10)

To a solution of Int-4 (4.5 g, 0.0084 mol) and Int-5 (488 mg, 0.00105 mol) in toluene (140 mL, 31 V), was added PTSA monohydride (2 g, 0.0105 mol) at 25-30° C. Resulting reaction mixture was refluxed at 130° C. for 18 h in a dean-stark apparatus. Progress of the reaction was monitored by TLC. After completion of reaction, reaction mixture was evaporated to remove toluene, residue obtained was treated with sodium bicarbonate solution and product was extracted with Ethyl acetate (3×100 mL). Organic layer was dried over Na₂SO₄ and concentrated under reduced pressure to get the crude. Crude was purified through combi flash, compound was eluted at 40-50% Ethyl acetate:Hexane. Pure fractions were evaporated to afford (2-{4-[(8Z,11Z)-heptadeca-8,11-dien-1-yl]-5-heptadecyl-1,3-dioxolan-2-yl}ethyl)dimethylamine (Lipid-10) as brown liquid.

Yield: 2 g (38.4%); Characterized by 1H-NMR and LCMS

LCMS: (EI, m/z) calcd for C₄₁H₇₉NO₂ [M+H]: 618.8; HPLC Purity: 74.09%

¹H NMR (400 MHZ, CDCl₃): 5.40-5.32 (4H, m), 5.04-5.01 (1H, t, J=4.8 Hz), 3.56-3.55 (2H, m), 2.79-2.76 (2H, t, J=6.4 Hz), 2.58-2.40 (2H, m), 2.31 (6H, s), 2.08-2.03 (4H, q, J₁=13.6 Hz, J₂=13.6 Hz), 1.89-1.87 (2H, t, J=3.2 Hz), 1.54-1.53 (4H, m), 1.44-1.26 (44H, m), 0.91-0.86 (6H, m)

### Example 11: Formulations comprising mRNA and sgRNA

Lipid nanoparticles (LNPs) were generated by rapid mixing. A mixture containing ionizable lipid, DSPC, cholesterol, and DMG-PEG at a molar ratio of 45:10:44:1, respectively, in ethanol was mixed with an aqueous sample containing Cas9 mRNA and synthetic sgRNA in 100 mM sodium acetate. Molar fractions were maintained for each formulation while varying the ionizable lipid. After formulation, particles immediately neutralized by dilution into Phosphate Buffered Saline (PBS). LNP size was determined using a Wyatt Plate Reader III with default settings.

Particles were functionally tested using amplicon sequencing. For testing, each formulation was dosed at 20 uL into 5,000 HEK293 FT or Jurkat cells. After a four day incubation cells were lysed and the locus of interested was amplified with PCR primers. These PCR products are then prepared for sequencing and sequenced on a MiSeq. The sequencing data is then processed and analyzed using CRISPR-DAV, which outputs indel percentages. (Table 1) For general reference, see Xuning Wang, Charles Tilford, Isaac Neuhaus, Gabe Mintier, Qi Guo, John N Feder, Stefan Kirov, CRISPR-DAV: CRISPR NGS data analysis and visualization pipeline, Bioinformatics, Volume 33, Issue 23, December 012017, Pages 3811-3812.

TABLE 1

| | | | average | average | Indel % | Indel | | |
|---|---|---|---|---|---|---|---|---|
| Ionizable | mRNA cargo | sgRNA | Radius | Radius | rep1 | % | Indel % | Indel |
| lipid | concentration | concentation | (nm) | (nm) | HEK293 | rep1 | rep2 | % rep2 |
| No. | (mg/ml) | (mg/mL) | rep1 | rep2 | FT | Jurkat | Hek | Jurkat |
| Lipid 11 | 0.087 | 0.087 | 122.675 | 109.475 | 93.59 | 70.3 | 95.87 | 65.07 |
| Lipid 4 | 0.087 | 0.087 | 153.05 | 150.55 | 53.08 | 34.09 | 62.46 | 19.69 |
| Lipid 8 | 0.087 | 0.087 | 135.075 | 146.35 | 67.32 | 2.26 | 74.04 | 0.37 |
| Lipid 3 | 0.087 | 0.087 | 92.55 | 91.525 | 92.23 | 77.31 | 90.92 | 66.37 |
| Lipid 9 | 0.087 | 0.087 | 105 | 101.525 | 84.41 | 15.29 | 66.39 | 10 |
| Lipid 10 | 0.087 | 0.087 | 177.3 | 129.475 | 51.22 | 1.75 | 63.9 | 1.04 |
| Lipid 2 | 0.087 | 0.087 | 142.825 | 136.875 | 16.5 | 0.15 | 41.5 | 0.09 |
| Lipid 7 | 0.087 | 0.087 | 116 | 124.85 | 2.58 | 0.06 | 2.29 | 0.02 |
| Lipid 6 | 0.087 | 0.087 | 98.825 | 91.175 | 30.63 | 13.16 | 8.87 | 18.09 |
| Lipid 5 | 0.087 | 0.087 | 94.25 | 90.275 | 0.09 | 0.11 | 0.12 | 0.1 |

We claim:

1. A compound selected from Table 1 or a pharmaceutically acceptable salt thereof:

| Compound | |
|---|---|
| No. | Structure |
| 1. | 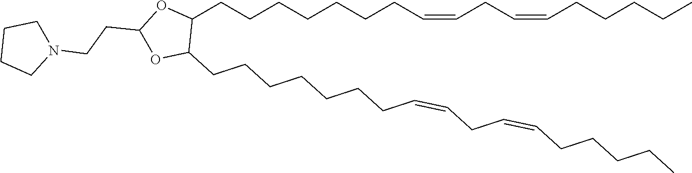 [SMILES: CCCCC/C=C\C/C=C\CCCCCCCC1OC(CCN2CCCC2)OC1CCCCCCC/C=C\C/C=C\CCCCC] |
| 2. | 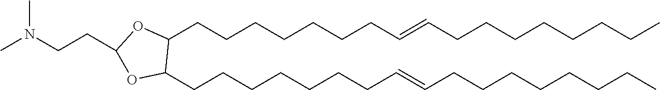 |
| 3. | 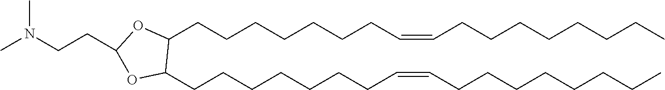 |
| 4. | 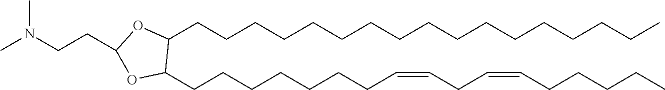 |
| 5. | 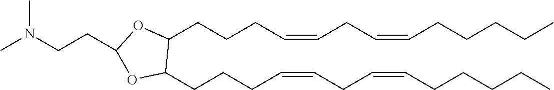 |
| 6. | 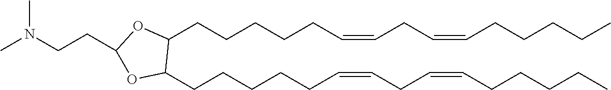 |
| 7. | 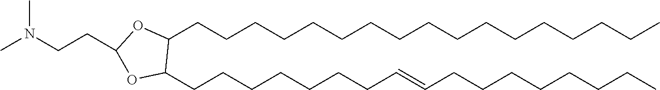 |
| 8. | 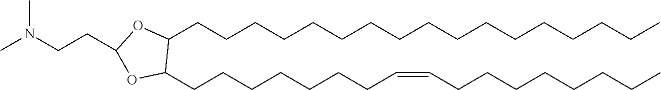 |
| 9. | 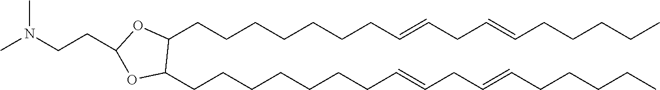 |
| 10. | 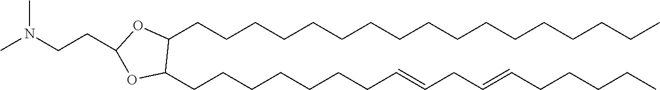 |

2. A lipid nanoparticle formulation comprising a compound according to claim 1 and optionally a PEG-modified lipid.

3. A lipid nanoparticle formulation according to claim 2 further comprising a cholesterol.

4. A lipid nanoparticle comprising a compound according to claim 2 further comprising a PEG-modified lipid, a non-cationic lipid and cholesterol.

5. The formulation according to claim 4 wherein said PEG-modified lipid is DMG-PEG.

6. The formulation according to claim 4 wherein said non-cationic lipid is selected from distearoylphosphatidylcholine (DSPC), dioleoylphosphatidylcholine (DOPC), dipalmitoylphosphatidylcholine (DPPC), diolcoylphosphatidylglycerol (DOPG), dipalmitoylphosphatidylglycerol (DPPG), diolcoylphosphatidylethanolamine (DOPE), palmitoyloleoylphosphatidylcholine (POPC), palmitoyloleoyl-phosphatidylethanolamine (POPE), diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

7. The formulation according to claim 4 wherein said non-cationic lipid is DOPE or DSPC.

8. A composition comprising a messenger RNA (mRNA) encoding a protein or a peptide, encapsulated within a lipid nanoparticle comprising a compound according to claim 1, a PEG-modified lipid and a non-cationic lipid.

9. The composition according to claim 8, wherein said lipid nanoparticle has a size less than about 150 nm or about 100 nm.

10. The composition according to claim 8 wherein said mRNA is Cas9 mRNA.

11. A method for delivery of mRNA for in vivo production of a protein or a peptide comprising administering to a subject a composition comprising an mRNA that encodes the protein or the peptide, wherein the mRNA is encapsulated within a lipid nanoparticle and wherein the administering of the composition results in the expression of the protein or the peptide encoded by the mRNA, wherein the lipid nanoparticle comprises, a PEG-modified lipid, a compound according to claim 1, a non-cationic lipid and cholesterol

12. The method according to claim 11, wherein the non-cationic lipid is selected from DOPE and DSPC.

13. The method according to claim 11, wherein said mRNA is Cas9 mRNA.

14. A process of encapsulating mRNA in lipid nanoparticles comprising a step of mixing (a) a solution comprising one or more mRNAs and one or more sgRNAs with (b) a lipid solution comprising one or more compounds of claim 1, one or more non-cationic lipids, and one or more PEG-modified lipids.

15. The process according to claim 14, wherein said lipid solution further comprises cholesterol.

16. A composition comprising a messenger RNA (mRNA) encoding a protein or a peptide, and an sgRNA encapsulated within a lipid nanoparticle comprising a compound selected from table below:

| | |
|---|---|
| 11 | 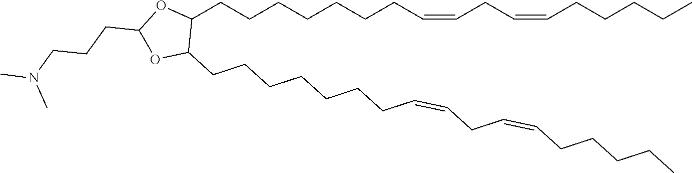 [SMILES: CCCCC/C=C\C/C=C\CCCCCCCC1OC(CCCN(C)C)OC1CCCCCCC/C=C\C/C=C\CCCCC] |
| 12 | 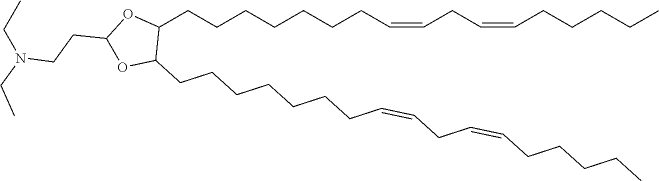 [SMILES: CCCCC/C=C\C/C=C\CCCCCCCC1OC(CCN(CC)CC)OC1CCCCCCC/C=C\C/C=C\CCCCC] |
| 13. | 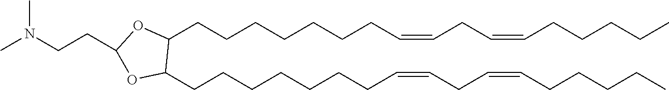 |

a PEG-modified lipid and a non-cationic lipid.

17. The composition according to claim 16, wherein said lipid nanoparticle has a size less than about 150 nm or about 100 nm.

18. The composition according to claim 16 wherein said mRNA is Cas9 mRNA.

19. A method for delivery of mRNA for in vivo production of a protein or a peptide comprising administering to a subject a composition comprising an mRNA that encodes the protein or the peptide, wherein the mRNA is encapsulated within a lipid nanoparticle and wherein the administering of the composition results in the expression of the protein or the peptide encoded by the mRNA, wherein the lipid nanoparticle comprises, a PEG-modified lipid, and sgRNA and a compound selected from table below:

| | |
|---|---|
| 11 | 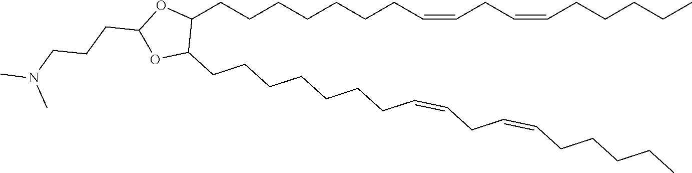 [SMILES: CCCCC/C=C\C/C=C\CCCCCCCC1OC(CCCN(C)C)OC1CCCCCCC/C=C\C/C=C\CCCCC] |
| 12 | 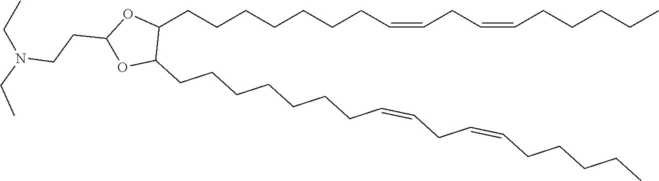 [SMILES: CCCCC/C=C\C/C=C\CCCCCCCC1OC(CCN(CC)CC)OC1CCCCCCC/C=C\C/C=C\CCCCC] |
| 13. | 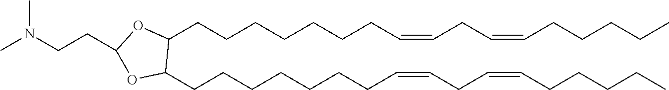 |

a non-cationic lipid and cholesterol.

20. The method according to claim 19, wherein the non-cationic lipid is selected from DOPE and DSPC.

21. The method according to claim 19, wherein said mRNA is Cas9 mRNA.

22. A process of encapsulating mRNA in lipid nanoparticles comprising a step of mixing (a) an mRNA solution comprising one or more mRNAs and an sgRNA with (b) a lipid solution comprising one or more compounds selected from table below:

| | |
|---|---|
| 11 | 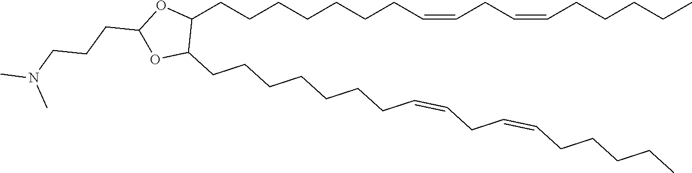 [SMILES: CCCCC/C=C\C/C=C\CCCCCCCC1OC(CCCN(C)C)OC1CCCCCCC/C=C\C/C=C\CCCCC] |
| 12 | 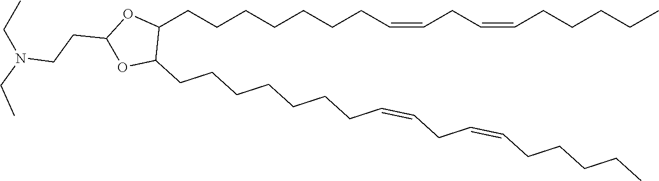 [SMILES: CCCCC/C=C\C/C=C\CCCCCCCC1OC(CCN(CC)CC)OC1CCCCCCC/C=C\C/C=C\CCCCC] |
| 13. | 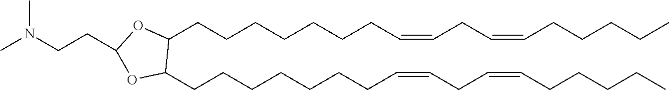 |

one or more non-cationic lipids, and one or more PEG-modified lipids.
